(19) United States
(12) Patent Application Publication
(10) Pub. No.: US 2016/0040236 A1
(43) Pub. Date: Feb. 11, 2016

(54) SYSTEMS AND METHODS FOR CHARACTERIZATION OF MULTIPLE SCLEROSIS

(71) Applicant: BIOGEN MA INC., Cambridge, MA (US)

(72) Inventors: Raghavendra Hosur, Cambridge, MA (US); Suzanne Szak, Milton, MA (US); Jadwiga Bienkowska, Cambridge, MA (US)

(21) Appl. No.: 14/776,436

(22) PCT Filed: Mar. 15, 2014
(86) PCT No.: PCT/US14/29925

§ 371 Date: Sep. 14, 2015

Related U.S. Application Data
(60) Provisional application No. 61/793,782, filed on Mar. 15, 2013.

(51) Int. Cl. C12Q 1/68; A61K 31/137; C07K 16/28; A61K 38/21; A61K 38/02

(52) U.S. Cl. CPC C12Q 1/6883; A61K 38/215; A61K 38/02; C07K 16/2866; A61K 31/137; C12Q 2600/106; C12Q 2600/158; C12Q 2600/118; C07K 2317/24; C07K 2317/76

(57) ABSTRACT

Methods and systems to characterize multiple sclerosis in a subject, e.g., in a subject a progressive form of MS are disclosed.

### CROSS REFERENCE TO RELATED APPLICATIONS

This application claims the benefit of U.S. Provisional Application No. 61/793,782, filed Mar. 15, 2013, the contents of which are incorporated herein by reference in their entirety.

### BACKGROUND OF THE INVENTION

Multiple sclerosis (MS) is an inflammatory disease of the brain and spinal cord characterized by recurrent foci of inflammation that lead to destruction of the myelin sheath. Each case of MS displays one of several patterns of presentation and subsequent course. Most commonly, MS first manifests itself as a series of attacks followed by complete or partial remissions as symptoms mysteriously lessen, only to return later after a period of stability. This is called relapsing-remitting (RR) MS. Primary-progressive (PP) MS is characterized by a gradual clinical decline with no distinct remissions, although there may be temporary plateaus or minor relief from symptoms. Secondary-progressive (SP) MS typically begins with a relapsing-remitting course followed by a later primary-progressive course. Rarely, patients may have a progressive-relapsing (PR) course in which the disease takes a progressive path punctuated by acute attacks. PP, SP, and PR are sometimes lumped together and called chronic progressive MS. A few patients experience malignant MS, defined as a swift and relentless decline resulting in significant disability or even death shortly after disease onset.

Currently, no therapy is effective against SPMS. One of the major reasons for this is an increased heterogeneity of disease presentation in SPMS patients. There is a need for improved identification and characterization of MS, including SPMS, patient populations.

### SUMMARY OF THE INVENTION

The invention relates, inter alia, to methods of treating and/or evaluating a subject having multiple sclerosis (MS), e.g., secondary progressive multiple sclerosis (SPMS), or at risk of developing MS, e.g., SPMS, methods of identifying a subject for treatment with a MS therapy, e.g., SPMS therapy, methods of treating or preventing one or more symptoms associated with MS, e.g., SPMS, and methods of evaluating or monitoring disease progression in a subject having MS, e.g., SPMS, or at risk of developing MS, e.g., SPMS. Systems for evaluating a subject population having MS, e.g., SPMS, and kits for identifying a subject for treatment with an MS therapy and/or clinical outcome are also described herein.

In one aspect, the present invention provides a method of treating and/or evaluating a subject having multiple sclerosis (MS), e.g., secondary progressive multiple sclerosis (SPMS), or at risk of developing MS, e.g., SPMS. The method includes administering an MS therapy, e.g., an MS therapy described herein, to a subject. In some embodiments, the subject has MS, e.g., SPMS, or is at risk of developing MS, e.g., SPMS, and the subject has one or more (e.g., 1, 2, 3, 4, 5, or 6) of the following: two or more (e.g., 2, 3, 4, 5, 6, 7, 8, 9, 10, 25, 50, 100, 250, 500, or more) genes associated with B cells differentially expressed (e.g., down-regulated), two or more (e.g., 2, 3, 4, 5, 6, 7, 8, 9, 10, 25, 50, 100, 250, 500, or more) genes associated with T cells differentially expressed (e.g., down-regulated), two or more (e.g., 2, 3, 4, 5, 6, 7, 8, 9, 10, 25, 50, 100, 250, 500, or more) genes associated with early erythrocytes (e-ERYs) differentially expressed (e.g., down-regulated), two or more (e.g., 2, 3, 4, 5, 6, 7, 8, 9, 10, 25, 50, 100, 250, 500, or more) genes associated with granulocyte/monocyte progenitors (GMPs) differentially expressed (e.g., up-regulated), two or more (e.g., 2, 3, 4, 5, 6, 7, 8, 9, 10, 25, 50, 100, 250, 500, or more) genes associated with dendritic cells (DCs) differentiated expressed (e.g., down-regulated or up-regulated), and/or two or more (e.g., 2, 3, 4, 5, 6, 7, 8, 9, 10, 25, 50, 100, 250, 500, or more) genes associated with natural killer (NK) cells differentiated expressed (e.g., down-regulated or up-regulated).

In some embodiments, the subject has MS, e.g., SPMS, or is at risk of developing MS, e.g., SPMS, and has two or more (e.g., 2, 3, 4, 5, 6, 7, 8, 9, 10, 11, 12, 13, 14, 15, 16, 17, 18, 19, 20, 30, 40, 50, or more) genes described herein differentially expressed (e.g., up-regulated or down-regulated). In some embodiments, the two or more (e.g., 2, 3, 4, 5, 6, 7, 8, 9, 10, 11, 12, 13, 14, or 15) differentially expressed genes are selected from, e.g., FCRL1, IGHM, 231418_PM_at, CD22, IGH@, 217138_PM_x_at, POU2AF1, LOC283663, MS4A1, IGL@, TCL1A, MS4A1, IGHD, CLLU1, and IGK@.

In some embodiments, the method includes acquiring knowledge and/or evaluating a sample to determine if a subject has one or more (e.g., 1, 2, 3, 4, 5, or 6) of the following: two or more (e.g., 2, 3, 4, 5, 6, 7, 8, 9, 10, 25, 50, 100, 250, 500, or more) genes associated with B cells differentially expressed (e.g., down-regulated), two or more (e.g., 2, 3, 4, 5, 6, 7, 8, 9, 10, 25, 50, 100, 250, 500, or more) genes associated with T cells differentially expressed (e.g., down-regulated), two or more (e.g., 2, 3, 4, 5, 6, 7, 8, 9, 10, 25, 50, 100, 250, 500, or more) genes associated with early erythrocytes (e-ERYs) differentially expressed (e.g., down-regulated), two or more (e.g., 2, 3, 4, 5, 6, 7, 8, 9, 10, 25, 50, 100, 250, 500, or more) genes associated with granulocyte/monocyte progenitors (GMPs) differentially expressed (e.g., up-regulated), two or more (e.g., 2, 3, 4, 5, 6, 7, 8, 9, 10, 25, 50, 100, 250, 500, or more) genes associated with DCs differentiated expressed (e.g., down-regulated or up-regulated), and/or two or more (e.g., 2, 3, 4, 5, 6, 7, 8, 9, 10, 25, 50, 100, 250, 500, or more) genes associated with NK cells differentiated expressed (e.g., down-regulated or up-regulated), and, based upon that knowledge, administering the subject an MS therapy, e.g., an MS therapy described herein.

In some embodiments, the gene associated with B cells is differentially expressed, e.g., down-regulated, by at least about 10%, 20%, 30%, 40%, 50%, 60%, 70%, 80%, 90%, 95%, or 99%, compared to a standard, e.g., an expression level of the gene in B cells in a normal subject. In some embodiments, the gene associated with T cells is differentially expressed, e.g., down-regulated, by at least about 10%, 20%, 30%, 40%, 50%, 60%, 70%, 80%, 90%, 95%, or 99%, compared to a standard, e.g., an expression level of the gene in T cells in a normal subject. In some embodiments, the gene associated with e-ERYs is differentially expressed, e.g., down-regulated, by at least about 10%, 20%, 30%, 40%, 50%, 60%, 70%, 80%, 90%, 95%, 99%, or 99.9%, compared to a standard, e.g., an expression level of the gene in e-ERYs in a normal subject. In some embodiments, the gene associated with GMPs is differentially expressed, e.g., up-regulated, by at least about 0.5, 1, 2, 5, 10, 20, 50, 100, 200, 500, or 1000 fold, compared to a standard, e.g., an expression level of the gene in GMPs in a normal subject. In some embodiments, the gene associated with DCs is differentially expressed, e.g., down-regulated, by at least about 10%, 20%, 30%, 40%, 50%, 60%, 70%, 80%, 90%, 95%, 99%, or 99.9%, or up-regulated, by at least about 0.5, 1, 2, 5, 10, 20, 50, 100, 200, 500, or 1000 fold, compared to a standard, e.g., an expression level of the gene in DCs in a normal subject. In some embodiments, the gene associated with NK cells is differentially expressed, e.g., down-regulated, by at least about 10%, 20%, 30%, 40%, 50%, 60%, 70%, 80%, 90%, 95%, 99%, or 99.9%, or up-regulated, by at least about 0.5, 1, 2, 5, 10, 20, 50, 100, 200, 500, or 1000 fold, compared to a standard, e.g., an expression level of the gene in NK cells in a normal subject.

In certain embodiments, the gene associated with B cells is a B cell-specific gene. In certain embodiments, the gene associated with T cells is a T cell-specific gene. In certain embodiments, the gene associated with e-ERYs is an e-ERY-specific gene. In certain embodiments, the gene associated with GMPs is a GMP-specific gene. In certain embodiments, the gene associated with DCs is a DC-specific gene. In certain embodiments, the gene associated with NK cells is a NK cell-specific gene.

In some embodiments, the method includes acquiring knowledge and/or evaluating a sample to determine if a subject has two or more (e.g., 2, 3, 4, 5, 6, 7, 8, 9, 10, 11, 12, 13, 14, 15, 16, 17, 18, 19, 20, 30, 40, 50, or more) genes described herein differentially expressed (e.g., up-regulated or down-regulated), and, based upon that knowledge, administering the subject an MS therapy, e.g., an MS therapy described herein. In some embodiments, the two or more (e.g., 2, 3, 4, 5, 6, 7, 8, 9, 10, 11, 12, 13, 14, or 15) differentially expressed genes are selected from, e.g., FCRL1, IGHM, 231418_PM_at, CD22, IGH@, 217138_PM_x_at, POU2AF1, LOC283663, MS4A1, IGL@, TCL1A, MS4A1, IGHD, CLLU1, and IGK@. In some embodiments, the two or more genes are differentially expressed, e.g., down-regulated, by at least about 10%, 20%, 30%, 40%, 50%, 60%, 70%, 80%, 90%, 95%, 99%, or 99.9%, or up-regulated, by at least about 0.5, 1, 2, 5, 10, 20, 50, 100, 200, 500, or 1000 fold, compared to a standard, e.g., an expression level of the same gene in the same cell type in a normal subject.

In particular embodiments, the MS therapy comprises an anti-VLA-4 therapy, e.g., natalizumab. In particular embodiments, the MS therapy comprises an anti-CD25 therapy, e.g., daclizumab. In particular embodiments, the MS therapy comprises an interferon beta, e.g., interferon beta-1a or interferon beta-1b. In particular embodiments, the MS therapy comprises a sphingosine 1-phosphate (S1P) antagonist, e.g., fingolimod. In particular embodiments, the MS therapy comprises glatiramer acetate (GA). In certain embodiments, the subject has been treated with an MS therapy, e.g., an alternative MS therapy.

In some embodiments, the method includes acquiring a sample, e.g., a blood sample, from the subject.

In certain embodiments, the method includes determining the expression levels of one or more (e.g., 1, 2, 3, 4, 5, or 6) of the following: two or more (e.g., 2, 3, 4, 5, 6, 7, 8, 9, 10, 25, 50, 100, 250, 500, or more) genes associated with B cells, two or more (e.g., 2, 3, 4, 5, 6, 7, 8, 9, 10, 25, 50, 100, 250, 500, or more) genes associated with T cells, two or more (e.g., 2, 3, 4, 5, 6, 7, 8, 9, 10, 25, 50, 100, 250, 500, or more) genes associated with e-ERYs, two or more (e.g., 2, 3, 4, 5, 6, 7, 8, 9, 10, 25, 50, 100, 250, 500, or more) genes associated with GMPs, two or more genes associated with DCs, and/or two or more genes associated with NK cells, in the sample.

In some embodiments, the method includes determining the expression levels of two or more (e.g., 2, 3, 4, 5, 6, 7, 8, 9, 10, 11, 12, 13, 14, 15, 16, 17, 18, 19, 20, 30, 40, 50, or more) genes described herein in the sample. In some embodiments, the two or more (e.g., 2, 3, 4, 5, 6, 7, 8, 9, 10, 11, 12, 13, 14, or 15) genes are selected from, e.g., FCRL1, IGHM, 231418_PM_at, CD22, IGH@, 217138_PM_x_at, POU2AF1, LOC283663, MS4A1, IGL@, TCL1A, MS4A1, IGHD, CLLU1, and IGK@.

In particular embodiments, the expression levels are determined prior to initiating, during, or after, a treatment in the subject. In some embodiments, the expression levels are determined at the time of diagnosis of the subject with MS, e.g., SPMS.

In some embodiments, the expression levels of the genes are determined by a method described herein, e.g., oligonucleotide array, an immunoassay (e.g., immunohistochemistry, northern blot, or a PCR method (e.g., quantitative RT-PCR). In some embodiments, expression levels of the genes are determined by evaluating the level of protein expression, e.g., by an immunoassay, e.g., by ELISA, immunohistochemistry, immunofluorescence, or western blot.

In some embodiments, the method includes comparing the expression levels of the genes with a standard, e.g., an expression level of the same gene in the same cell type in a normal subject.

In some embodiments, the method includes identifying a subject having one or more (e.g., 1, 2, 3, 4, 5, or 6) of the following: two or more (e.g., 2, 3, 4, 5, 6, 7, 8, 9, 10, 25, 50, 100, 250, 500, or more) genes associated with B cells differentially expressed (e.g., down-regulated, two or more (e.g., 2, 3, 4, 5, 6, 7, 8, 9, 10, 25, 50, 100, 250, 500, or more) genes associated with T cells differentially expressed (e.g., down-regulated), two or more (e.g., 2, 3, 4, 5, 6, 7, 8, 9, 10, 25, 50, 100, 250, 500, or more) genes associated with e-ERYs differentially expressed (e.g., down-regulated), two or more (e.g., 2, 3, 4, 5, 6, 7, 8, 9, 10, 25, 50, 100, 250, 500, or more) genes associated with GMPs differentially expressed (e.g., up-regulated), two or more (e.g., 2, 3, 4, 5, 6, 7, 8, 9, 10, 25, 50, 100, 250, 500, or more) genes associated with DCs differentiated expressed (e.g., down-regulated or up-regulated), and/or two or more (e.g., 2, 3, 4, 5, 6, 7, 8, 9, 10, 25, 50, 100, 250, 500, or more) genes associated with NK cells differentiated expressed (e.g., down-regulated or up-regulated), for treatment with an MS therapy, e.g., an MS therapy described herein.

In some embodiments, the method includes identifying a subject having two or more (e.g., 2, 3, 4, 5, 6, 7, 8, 9, 10, 11, 12, 13, 14, 15, 16, 17, 18, 19, 20, 30, 40, 50, or more) genes described herein differentially expressed (e.g., up-regulated or down-regulated), for treatment with an MS therapy, e.g., an MS therapy described herein. In some embodiments, the two or more (e.g., 2, 3, 4, 5, 6, 7, 8, 9, 10, 11, 12, 13, 14, or 15) differentially expressed genes are selected from, e.g., FCRL1, IGHM, 231418_PM_at, CD22, IGH@, 217138_PM_x_at, POU2AF1, LOC283663, MS4A1, IGL@, TCL1A, MS4A1, IGHD, CLLU1, and IGK@.

In certain embodiments, the subject is already receiving an MS therapy, e.g., an MS therapy described herein, and the identification of the differential expression (e.g., down-regulation or up-regulation) of the genes indicates that the subject can receive an alternative MS therapy, e.g., an alternative MS therapy described herein. In certain embodiments, the subject is already receiving an MS therapy, e.g., an MS therapy described herein, and the identification of the differential expression (e.g., down-regulation or the up-regulation) of the genes indicates that the subject should stop receiving the MS therapy, or the dose or dosing schedule of the MS therapy should be altered, e.g., reduced or increased.

In some embodiments, the method includes identifying a clinical outcome (e.g., disease severity, disease progression, clinical outcome, or prognosis) of the subject having one or more (e.g., 1, 2, 3, 4, 5, or 6) of the following: two or more (e.g., 2, 3, 4, 5, 6, 7, 8, 9, 10, 25, 50, 100, 250, 500, or more) genes associated with B cells, two of more (e.g., 2, 3, 4, 5, 6, 7, 8, 9, 10, 25, 50, 100, 250, 500, or more) genes associated with GMPs, two or more (e.g., 2, 3, 4, 5, 6, 7, 8, 9, 10, 25, 50, 100, 250, 500, or more) genes associate with ERYs, two or more (e.g., 2, 3, 4, 5, 6, 7, 8, 9, 10, 25, 50, 100, 250, 500, or more) genes associated with DCs, and/or two or more (e.g., 2, 3, 4, 5, 6, 7, 8, 9, 10, 25, 50, 100, 250, 500, or more) genes associated with NK cells, differentiated expressed (e.g., up-regulated or down-regulated), wherein the differential expression (e.g., up-regulation or down-regulation) is correlated with or indicative of a clinical score, e.g., a clinical score associated with disease severity, disease progression, clinical outcome, or prognosis, e.g., a clinical score described herein.

In some embodiments, the method includes identifying a clinical outcome (e.g., disease severity, disease progression, clinical outcome, or prognosis) of the subject having two or more (e.g., 2, 3, 4, 5, 6, 7, 8, 9, 10, 11, 12, 13, 14, 15, 16, 17, 18, 19, 20, 30, 40, 50, or more) genes described herein differentially expressed (e.g., up-regulated or down-regulated), for treatment with an MS therapy, e.g., an MS therapy described herein. In some embodiments, the two or more (e.g., 2, 3, 4, 5, 6, 7, 8, 9, 10, 11, 12, 13, 14, or 15) differentially expressed genes are selected from, e.g., FCRL1, IGHM, 231418_PM_at, CD22, IGH@, 217138_PM_x_at, POU2AF1, LOC283663, MS4A1, IGL@, TCL1A, MS4A1, IGHD, CLLU1, and IGK@.

In particular embodiments, the method includes selecting an MS therapy, e.g., an MS therapy described herein, for the subject. In some embodiments, the method includes determining a clinical score for the subject, e.g., a clinical score associated with disease severity, disease progression, clinical outcome, or prognosis, e.g., a clinical score described herein, e.g., Expanded Disability Status Scale (EDSS), or Multiple Sclerosis Severity Score (MSSS).

In some embodiments, the method includes selecting a subject having MS, e.g., SPMS, or at risk for MS, e.g., SPMS, for treatment with an MS therapy, e.g., an MS therapy described herein, based upon a determination of one or more (e.g., 1, 2, 3, 4, 5, or 6) of the following: two or more (e.g., 2, 3, 4, 5, 6, 7, 8, 9, 10, 25, 50, 100, 250, 500, or more) genes associated with B cells are differentially expressed (e.g., down-regulated), two or more (e.g., 2, 3, 4, 5, 6, 7, 8, 9, 10, 25, 50, 100, 250, 500, or more) genes associated with T cells are differentially expressed (e.g., down-regulated), two or more (e.g., 2, 3, 4, 5, 6, 7, 8, 9, 10, 25, 50, 100, 250, 500, or more) genes associated with early erythrocytes (e-ERYs) are differentially expressed (e.g., down-regulated), two or more (e.g., 2, 3, 4, 5, 6, 7, 8, 9, 10, 25, 50, 100, 250, 500, or more) genes associated with granulocyte/monocyte progenitors (GMPs) are differentially expressed (e.g., up-regulated), two or more (e.g., 2, 3, 4, 5, 6, 7, 8, 9, 10, 25, 50, 100, 250, 500, or more) genes associated with DCs are differentially expressed (e.g., down-regulated or up-regulated), and/or two or more (e.g., 2, 3, 4, 5, 6, 7, 8, 9, 10, 25, 50, 100, 250, 500, or more) genes associated with NK cells are differentiated expressed (e.g., down-regulated or up-regulated).

In some embodiments, the method includes selecting a subject having MS, e.g., SPMS, or at risk for MS, e.g., SPMS, for treatment with an MS therapy, e.g., an MS therapy described herein, based upon a determination that two or more (e.g., 2, 3, 4, 5, 6, 7, 8, 9, 10, 11, 12, 13, 14, 15, 16, 17, 18, 19, 20, 30, 40, 50, or more) genes described herein are differentially expressed (e.g., up-regulated or down-regulated). In some embodiments, the two or more (e.g., 2, 3, 4, 5, 6, 7, 8, 9, 10, 11, 12, 13, 14, or 15) differentially expressed genes are selected from, e.g., FCRL1, IGHM, 231418_PM_at, CD22, IGH@, 217138_PM_x_at, POU2AF1, LOC283663, MS4A1, IGL@, TCL1A, MS4A1, IGHD, CLLU1, and IGK@.

In another aspect, the present invention provides a method of treating and/or evaluating a subject having multiple sclerosis (MS), e.g., secondary progressive multiple sclerosis (SPMS), or at risk of developing MS, e.g., SPMS. In some embodiments, the method includes administering an MS therapy, e.g., an MS therapy described herein, to a subject. In some embodiments, the subject has MS, e.g., SPMS, or is at risk of developing MS, e.g., SPMS, and the subject has one or more (e.g., 1, 2, 3, 4, 5, or 6) of the following: two or more (e.g., 2, 3, 4, 5, 6, 7, 8, 9, 10, 25, 50, 100, 250, 500, or more) genes associated with B cells differentially expressed (e.g., up-regulated), two or more (e.g., 2, 3, 4, 5, 6, 7, 8, 9, 10, 25, 50, 100, 250, 500, or more) genes associated with T cells differentially expressed (e.g., up-regulated), two or more (e.g., 2, 3, 4, 5, 6, 7, 8, 9, 10, 25, 50, 100, 250, 500, or more) genes associated with early erythrocytes (e-ERYs) differentially expressed (e.g., up-regulated), two or more (e.g., 2, 3, 4, 5, 6, 7, 8, 9, 10, 25, 50, 100, 250, 500, or more) genes associated with granulocyte/monocyte progenitors (GMPs) differentially expressed (e.g., down-regulated), two or more (e.g., 2, 3, 4, 5, 6, 7, 8, 9, 10, 25, 50, 100, 250, 500, or more) genes associated with dendritic cells (DCs) differentiated expressed (e.g., down-regulated or up-regulated), and/or two or more (e.g., 2, 3, 4, 5, 6, 7, 8, 9, 10, 25, 50, 100, 250, 500, or more) genes associated with natural killer (NK) cells differentiated expressed (e.g., down-regulated or up-regulated).

In some embodiments, the subject has MS, e.g., SPMS, or is at risk of developing MS, e.g., SPMS, and has two or more (e.g., 2, 3, 4, 5, 6, 7, 8, 9, 10, 11, 12, 13, 14, 15, 16, 17, 18, 19, 20, 30, 40, 50, or more) genes described herein differentially expressed (e.g., up-regulated or down-regulated). In some embodiments, the two or more (e.g., 2, 3, 4, 5, 6, 7, 8, 9, 10, 11, 12, 13, 14, or 15) differentially expressed genes are selected from, e.g., FCRL1, IGHM, 231418_PM_at, CD22, IGH@, 217138_PM_x_at, POU2AF1, LOC283663, MS4A1, IGL@, TCL1A, MS4A1, IGHD, CLLU1, and IGK@.

In some embodiments, the method includes acquiring knowledge that and/or evaluating a sample to determine if, a subject has one or more of the following: two or more (e.g., 2, 3, 4, 5, 6, 7, 8, 9, 10, 25, 50, 100, 250, 500, or more) genes associated with B cells differentially expressed (e.g., up-regulated), two or more (e.g., 2, 3, 4, 5, 6, 7, 8, 9, 10, 25, 50, 100, 250, 500, or more) genes associated with T cells differentially expressed (e.g., up-regulated), two or more (e.g., 2, 3, 4, 5, 6, 7, 8, 9, 10, 25, 50, 100, 250, 500, or more) genes associated with early erythrocytes (e-ERYs) differentially expressed (e.g., up-regulated), two or more (e.g., 2, 3, 4, 5, 6, 7, 8, 9, 10, 25, 50, 100, 250, 500, or more) genes associated with granulocyte/monocyte progenitors (GMPs) differentially expressed (e.g., down-regulated), two or more (e.g., 2, 3, 4, 5, 6, 7, 8, 9, 10, 25, 50, 100, 250, 500, or more) genes associated with DCs differentiated expressed (e.g., down-regulated or up-regulated), and/or two or more (e.g., 2, 3, 4, 5, 6, 7, 8, 9, 10, 25, 50, 100, 250, 500, or more) genes associated with NK cells differentiated expressed (e.g., down-regulated or up-regulated), and, based upon that knowledge, administering the subject an MS therapy, e.g., an MS therapy described herein.

In some embodiments, the gene associated with B cells is differentially expressed, e.g., up-regulated, by at least about 0.5, 1, 2, 5, 10, 20, 50, 100, 200, 500, or 1000 fold, compared to a standard, e.g., an expression level of the gene in B cells in a normal subject. In some embodiments, the gene associated with T cells is differentially expressed, e.g., up-regulated, by at least about 0.5, 1, 2, 5, 10, 20, 50, 100, 200, 500, or 1000 fold, compared to a standard, e.g., an expression level of the gene in T cells in a normal subject. In some embodiments, the gene associated with e-ERYs is differentially expressed, e.g., up-regulated, by at least about 0.5, 1, 2, 5, 10, 20, 50, 100, 200, 500, or 1000 fold, compared to a standard, e.g., an expression level of the gene in e-ERYs in a normal subject. In some embodiments, the gene associated with GMPs is differentially expressed, e.g., down-regulated, by at least about 10%, 20%, 30%, 40%, 50%, 60%, 70%, 80%, 90%, 95%, or 99%, compared to a standard, e.g., an expression level of the gene in GMPs in a normal subject. In some embodiments, the gene associated with DCs is differentially expressed, e.g., down-regulated by at least about 10%, 20%, 30%, 40%, 50%, 60%, 70%, 80%, 90%, 95%, 99%, or 99.9%, or up-regulated by at least about 0.5, 1, 2, 5, 10, 20, 50, 100, 200, 500, or 1000 fold, compared to a standard, e.g., an expression level of the gene in DCs in a normal subject. In some embodiments, the gene associated with NK cells is differentially expressed, e.g., up-regulated, by at least about 0.5, 1, 2, 5, 10, 20, 50, 100, 200, 500, or 1000 fold, or down-regulated, by at least about 10%, 20%, 30%, 40%, 50%, 60%, 70%, 80%, 90%, 95%, 99%, or 99.9%, compared to a standard, e.g., an expression level of the gene in NK cells in a normal subject.

In certain embodiments, the gene associated with B cells is a B cell-specific gene. In certain embodiments, the gene associated with T cells is a T cell-specific gene. In certain embodiments, the gene associated with e-ERYs is an e-ERY-specific gene. In certain embodiments, the gene associated with GMPs is a GMP-specific gene. In certain embodiments, the gene associated with GCs is a GC-specific gene. In certain embodiments, the gene associated with NK cells is a NK cell-specific gene.

In some embodiments, the method includes acquiring knowledge and/or evaluating a sample to determine if a subject has two or more (e.g., 2, 3, 4, 5, 6, 7, 8, 9, 10, 11, 12, 13, 14, 15, 16, 17, 18, 19, 20, 30, 40, 50, or more) genes described herein differentially expressed (e.g., up-regulated or down-regulated), and, based upon that knowledge, administering the subject an MS therapy, e.g., an MS therapy described herein. In some embodiments, the two or more (e.g., 2, 3, 4, 5, 6, 7, 8, 9, 10, 11, 12, 13, 14, or 15) differentially expressed genes are selected from, e.g., FCRL1, IGHM, 231418_PM_at, CD22, IGH@, 217138_PM_x_at, POU2AF1, LOC283663, MS4A1, IGL@, TCL1A, MS4A1, IGHD, CLLU1, and IGK@. In some embodiments, the two or more genes are differentially expressed, e.g., down-regulated, by at least about 10%, 20%, 30%, 40%, 50%, 60%, 70%, 80%, 90%, 95%, 99%, or 99.9%, or up-regulated, by at least about 0.5, 1, 2, 5, 10, 20, 50, 100, 200, 500, or 1000 fold, compared to a standard, e.g., an expression level of the same gene in the same cell type in a normal subject.

In particular embodiments, the MS therapy comprises an anti-VLA-4 therapy, e.g., natalizumab. In particular embodiments, the MS therapy comprises an anti-IL-2 receptor therapy, e.g., daclizumab. In particular embodiments, the MS therapy comprises an interferon beta, e.g., interferon beta-1a or interferon beta-1b. In particular embodiments, the MS therapy comprises a sphingosine 1-phosphate (SIP) antagonist, e.g., fingolimod. In particular embodiments, the MS therapy comprises glatiramer acetate (GA). In some embodiments, the subject has been treated with an MS therapy, e.g., an alternative MS therapy.

In some embodiments, the method includes acquiring a sample, e.g., a blood sample, from the subject. In some embodiments, the method includes determining one or more (e.g., 1, 2, 3, 4, 5, or 6) of the following: the expression levels of two or more (e.g., 2, 3, 4, 5, 6, 7, 8, 9, 10, 25, 50, 100, 250, 500, or more) genes associated with B cells, two or more (e.g., 2, 3, 4, 5, 6, 7, 8, 9, 10, 25, 50, 100, 250, 500, or more) genes associated with T cells, two or more (e.g., 2, 3, 4, 5, 6, 7, 8, 9, 10, 25, 50, 100, 250, 500, or more) genes associated with e-ERYs, two or more (e.g., 2, 3, 4, 5, 6, 7, 8, 9, 10, 25, 50, 100, 250, 500, or more) genes associated with GMPs, two or more (e.g., 2, 3, 4, 5, 6, 7, 8, 9, 10, 25, 50, 100, 250, 500, or more) genes associated with DCs, and/or two or more (e.g., 2, 3, 4, 5, 6, 7, 8, 9, 10, 25, 50, 100, 250, 500, or more) genes associated with NK cells, in the sample.

In some embodiments, the method includes determining the expression levels of two or more (e.g., 2, 3, 4, 5, 6, 7, 8, 9, 10, 11, 12, 13, 14, 15, 16, 17, 18, 19, 20, 30, 40, 50, or more) genes described herein in the sample. In some embodiments, the two or more (e.g., 2, 3, 4, 5, 6, 7, 8, 9, 10, 11, 12, 13, 14, or 15) genes are selected from, e.g., FCRL1, IGHM, 231418_PM_at, CD22, IGH@, 217138_PM_x_at, POU2AF1, LOC283663, MS4A1, IGL@, TCL1A, MS4A1, IGHD, CLLU1, and IGK@.

In certain embodiments, the expression levels are determined prior to initiating, during, or after, a treatment in the subject. In certain embodiments, the expression levels are determined at the time of diagnosis of the subject with MS, e.g., SPMS.

In some embodiments, the expression levels of the genes are determined by a method described herein, e.g., oligonucleotide array, an immunoassay (e.g., immunohistochemistry, northern blot, or a PCR method (e.g., quantitative RT-PCR). In some embodiments, expression levels of the genes are determined by evaluating the level of protein expression, e.g., by an immunoassay, e.g., by ELISA, immunohistochemistry, immunofluorescence, or western blot.

In some embodiments, the method includes comprising comparing the expression levels of the genes with a standard, e.g., an expression level of the same gene in the same cell type in a normal subject.

In some embodiments, the method includes identifying a subject having one or more (e.g., 1, 2, 3, 4, 5, or 6) of the following: two or more (e.g., 2, 3, 4, 5, 6, 7, 8, 9, 10, 25, 50, 100, 250, 500, or more) genes associated with B cells differentially expressed (e.g., up-regulated), two or more (e.g., 2, 3, 4, 5, 6, 7, 8, 9, 10, 25, 50, 100, 250, 500, or more) genes associated with T cells differentially expressed (e.g., up-regulated), two or more (e.g., 2, 3, 4, 5, 6, 7, 8, 9, 10, 25, 50, 100, 250, 500, or more) genes associated with early erythrocytes (e-ERYs) differentially expressed (e.g., up-regulated), and two or more (e.g., 2, 3, 4, 5, 6, 7, 8, 9, 10, 25, 50, 100, 250, 500, or more) genes associated with granulocyte/monocyte progenitors (GMPs) differentially expressed (e.g., down-regulated), two or more (e.g., 2, 3, 4, 5, 6, 7, 8, 9, 10, 25, 50, 100, 250, 500, or more) genes associated with DCs differentiated expressed (e.g., down-regulated or up-regulated), and/or two or more (e.g., 2, 3, 4, 5, 6, 7, 8, 9, 10, 25, 50, 100, 250, 500, or more) genes associated with NK cells differentiated expressed (e.g., down-regulated or up-regulated), for treatment with an MS therapy, e.g., an MS therapy described herein.

In some embodiments, the method includes identifying a subject having two or more (e.g., 2, 3, 4, 5, 6, 7, 8, 9, 10, 11, 12, 13, 14, 15, 16, 17, 18, 19, 20, 30, 40, 50, or more) genes described herein differentially expressed (e.g., down-regulated or up-regulated), for treatment with an MS therapy, e.g., an MS therapy described herein. In some embodiments, the two or more (e.g., 2, 3, 4, 5, 6, 7, 8, 9, 10, 11, 12, 13, 14, or 15) differentially expressed genes are selected from, e.g., FCRL1, IGHM, 231418_PM_at, CD22, IGH@, 217138_PM_x_at, POU2AF1, LOC283663, MS4A1, IGL@, TCL1A, MS4A1, IGHD, CLLU1, and IGK@.

In certain embodiments, the subject is already receiving an MS therapy, e.g., an MS therapy described herein, and the identification of the differential expression (e.g., down-regulation or up-regulation) of the genes indicates that the subject can receive an alternative MS therapy, e.g., an alternative MS therapy described herein. In certain embodiments, the subject is already receiving an MS therapy, e.g., an MS therapy described herein, and the identification of the differential expression (e.g., down-regulation or the up-regulation) of the genes indicates that the subject should stop receiving the MS therapy or should receive an alternative MS therapy, or the dose or dosing schedule of the MS therapy should be altered, e.g., reduced or increased.

In some embodiments, the method includes identifying a clinical outcome (e.g., disease severity, disease progression, clinical outcome, or prognosis) of the subject having one or more (e.g., 1, 2, 3, 4, 5, or 6) of the following: two or more (e.g., 2, 3, 4, 5, 6, 7, 8, 9, 10, 25, 50, 100, 250, 500, or more) genes associated with B cells differentially expressed (e.g., up-regulated), two or more (e.g., 2, 3, 4, 5, 6, 7, 8, 9, 10, 25, 50, 100, 250, 500, or more) genes associated with T cells differentially expressed (e.g., up-regulated), two or more (e.g., 2, 3, 4, 5, 6, 7, 8, 9, 10, 25, 50, 100, 250, 500, or more) genes associated with early erythrocytes (e-ERYs) differentially expressed (e.g., up-regulated), two or more (e.g., 2, 3, 4, 5, 6, 7, 8, 9, 10, 25, 50, 100, 250, 500, or more) genes associated with granulocyte/monocyte progenitors (GMPs) differentially expressed (e.g., down-regulated), two or more (e.g., 2, 3, 4, 5, 6, 7, 8, 9, 10, 25, 50, 100, 250, 500, or more) genes associated with DCs differentiated expressed (e.g., down-regulated or up-regulated), and/or two or more (e.g., 2, 3, 4, 5, 6, 7, 8, 9, 10, 25, 50, 100, 250, 500, or more) genes associated with NK cells differentiated expressed (e.g., down-regulated or up-regulated), wherein the differential expression (e.g., up-regulation or down-regulation) is correlated with or indicative of a clinical score, e.g., a clinical score associated with disease severity, disease progression, clinical outcome, or prognosis, e.g., a clinical score described herein.

In some embodiments, the method includes identifying a clinical outcome (e.g., disease severity, disease progression, clinical outcome, or prognosis) of the subject having two or more (e.g., 2, 3, 4, 5, 6, 7, 8, 9, 10, 11, 12, 13, 14, 15, 16, 17, 18, 19, 20, 30, 40, 50, or more) genes described herein differentially expressed (e.g., up-regulated or down-regulated), wherein the differential expression (e.g., up-regulation or down-regulation) is correlated with or indicative of a clinical score, e.g., a clinical score associated with disease severity, disease progression, clinical outcome, or prognosis, e.g., a clinical score described herein. In some embodiments, the two or more (e.g., 2, 3, 4, 5, 6, 7, 8, 9, 10, 11, 12, 13, 14, or 15) differentially expressed genes are selected from, e.g., FCRL1, IGHM, 231418_PM_at, CD22, IGH@, 217138_PM_x_at, POU2AF1, LOC283663, MS4A1, IGL@, TCL1A, MS4A1, IGHD, CLLU1, and IGK@.

In some embodiments, the method includes selecting an MS therapy, e.g., an MS therapy described herein, for the subject.

In certain embodiments, the method includes determining a clinical score for the subject, e.g., a clinical score associated with disease severity, disease progression, clinical outcome, or prognosis, e.g., a clinical score described herein, e.g., Expanded Disability Status Scale (EDSS), or Multiple Sclerosis Severity Score (MSSS).

In some embodiments, the method includes selecting a subject having MS, e.g., SPMS, or at risk for MS, e.g., SPMS, for treatment with an MS therapy, e.g., an MS therapy described herein, based upon a determination of one or more (e.g., 1, 2, 3, 4, 5, or 6) of the following: two or more (e.g., 2, 3, 4, 5, 6, 7, 8, 9, 10, 25, 50, 100, 250, 500, or more) genes associated with B cells are differentially expressed (e.g., up-regulated), two or more (e.g., 2, 3, 4, 5, 6, 7, 8, 9, 10, 25, 50, 100, 250, 500, or more) genes associated with T cells are differentially expressed (e.g., up-regulated), two or more (e.g., 2, 3, 4, 5, 6, 7, 8, 9, 10, 25, 50, 100, 250, 500, or more) genes associated with early erythrocytes (e-ERYs) are differentially expressed (e.g., up-regulated), two or more (e.g., 2, 3, 4, 5, 6, 7, 8, 9, 10, 25, 50, 100, 250, 500, or more) genes associated with granulocyte/monocyte progenitors (GMPs) are differentially expressed (e.g., down-regulated), two or more (e.g., 2, 3, 4, 5, 6, 7, 8, 9, 10, 25, 50, 100, 250, 500, or more) genes associated with DCs are differentiated expressed (e.g., down-regulated or up-regulated), and/or two or more (e.g., 2, 3, 4, 5, 6, 7, 8, 9, 10, 25, 50, 100, 250, 500, or more) genes associated with NK cells are differentiated expressed (e.g., down-regulated or up-regulated).

In some embodiments, the method includes selecting a subject having MS, e.g., SPMS, or at risk for MS, e.g., SPMS, for treatment with an MS therapy, e.g., an MS therapy described herein, based upon a determination that two or more (e.g., 2, 3, 4, 5, 6, 7, 8, 9, 10, 11, 12, 13, 14, 15, 16, 17, 18, 19, 20, 30, 40, 50, or more) genes described herein differentially expressed (e.g., up-regulated or down-regulated), wherein the differential expression (e.g., up-regulation or down-regulation) is correlated with or indicative of a clinical score or clinical marker, e.g., a clinical score or clinical marker associated with disease severity, disease progression, clinical outcome, or prognosis, e.g., a clinical score or clinical marker described herein. In some embodiments, the two or more (e.g., 2, 3, 4, 5, 6, 7, 8, 9, 10, 11, 12, 13, 14, or 15) differentially expressed genes are selected from, e.g., FCRL1, IGHM, 231418_PM_at, CD22, IGH@, 217138_PM_x_at, POU2AF1, LOC283663, MS4A1, IGL@, TCL1A, MS4A1, IGHD, CLLU1, and IGK@.

In yet another aspect, the present invention provides a method of identifying a subject having multiple sclerosis (MS), e.g., secondary progressive multiple sclerosis (SPMS), or at risk of developing MS, e.g., SPMS, for treatment with an MS therapy, e.g., an MS therapy described herein. In some embodiments, the method includes providing a sample, e.g., a blood sample, from a subject having multiple sclerosis (MS), e.g., secondary progressive multiple sclerosis (SPMS), or at risk of developing MS, e.g., SPMS, determining the expression levels of one or more (e.g., 1, 2, 3, 4, 5, or 6) of the following: two or more (e.g., 2, 3, 4, 5, 6, 7, 8, 9, 10, 25, 50, 100, 250, 500, or more) genes associated with B cells, two or more (e.g., 2, 3, 4, 5, 6, 7, 8, 9, 10, 25, 50, 100, 250, 500, or more) genes associated with T cells, two or more (e.g., 2, 3, 4, 5, 6, 7, 8, 9, 10, 25, 50, 100, 250, 500, or more) genes associated with e-ERYs, two or more (e.g., 2, 3, 4, 5, 6, 7, 8, 9, 10, 25, 50, 100, 250, 500, or more) genes associated with GMPs, two or more (e.g., 2, 3, 4, 5, 6, 7, 8, 9, 10, 25, 50, 100, 250, 500, or more) genes associated with DCs, and/or two or more (e.g., 2, 3, 4, 5, 6, 7, 8, 9, 10, 25, 50, 100, 250, 500, or more) genes associated with NK cells, in the sample; comparing the expression levels of the genes with a standard, e.g., an expression level of the same gene in the same cell type in a normal subject; and identifying the subject for treatment with an MS therapy, e.g., an MS therapy described herein, on the basis that the subject has one or more (e.g., 1, 2, 3, 4, 5, or 6) of the following: two or more (e.g., 2, 3, 4, 5, 6, 7, 8, 9, 10, 25, 50, 100, 250, 500, or more) genes associated with B cells differentially expressed (e.g., down-regulated), two or more (e.g., 2, 3, 4, 5, 6, 7, 8, 9, 10, 25, 50, 100, 250, 500, or more) genes associated with T cells differentially expressed (e.g., down-regulated), two or more (e.g., 2, 3, 4, 5, 6, 7, 8, 9, 10, 25, 50, 100, 250, 500, or more) genes associated with e-ERYs differentially expressed (e.g., down-regulated), two or more (e.g., 2, 3, 4, 5, 6, 7, 8, 9, 10, 25, 50, 100, 250, 500, or more) genes associated with GMPs differentially expressed (e.g., up-regulated), two or more (e.g., 2, 3, 4, 5, 6, 7, 8, 9, 10, 25, 50, 100, 250, 500, or more) genes associated with DCs differentially expressed (e.g., down-regulated or up-regulated), and/or two or more (e.g., 2, 3, 4, 5, 6, 7, 8, 9, 10, 25, 50, 100, 250, 500, or more) genes associated with NK cells differentially expressed (e.g., down-regulated or up-regulated).

In some embodiments, the method includes determining the expression levels of two or more (e.g., 2, 3, 4, 5, 6, 7, 8, 9, 10, 11, 12, 13, 14, 15, 16, 17, 18, 19, 20, 30, 40, 50, or more) genes described herein in the sample; comparing the expression levels of the genes with a standard, e.g., an expression level of the same gene in the same cell type in a normal subject; and identifying the subject for treatment with an MS therapy, e.g., an MS therapy described herein, on the basis that the subject has two or more of the genes differentially expressed (e.g., down-regulated or up-regulated). In some embodiments, the two or more (e.g., 2, 3, 4, 5, 6, 7, 8, 9, 10, 11, 12, 13, 14, or 15) genes are selected from, e.g., FCRL1, IGHM, 231418_PM_at, CD22, IGH@, 217138_PM_x_at, POU2AF1, LOC283663, MS4A1, IGL@, TCL1A, MS4A1, IGHD, CLLU1, and IGK@.

In some embodiments, the expression levels of the genes are determined by a method described herein, e.g., oligonucleotide array, an immunoassay (e.g., immunohistochemistry, northern blot, or a PCR method (e.g., quantitative RT-PCR). In some embodiments, expression levels of the genes are determined by evaluating the level of protein expression, e.g., by an immunoassay, e.g., by ELISA, immunohistochemistry, immunofluorescence, or western blot.

In certain embodiments, the expression levels are determined prior to initiating, during, or after, a treatment in the subject. In certain embodiments, the expression levels are determined at the time of diagnosis of the subject with MS, e.g., SPMS.

In some embodiments, the method includes comprising comparing the expression levels of the genes with a standard, e.g., an expression level of the same gene in the same cell type in a normal subject.

In some embodiments, the gene associated with B cells is differentially expressed, e.g., down-regulated, by at least about 10%, 20%, 30%, 40%, 50%, 60%, 70%, 80%, 90%, 95%, or 99%, compared to a standard, e.g., an expression level of the gene in B cells in a normal subject. In some embodiments, the gene associated with T cells is differentially expressed, e.g., down-regulated, by at least about 10%, 20%, 30%, 40%, 50%, 60%, 70%, 80%, 90%, 95%, or 99%, compared to a standard, e.g., an expression level of the gene in T cells in a normal subject. In some embodiments, the gene associated with e-ERYs is differentially expressed, e.g., down-regulated, by at least about 10%, 20%, 30%, 40%, 50%, 60%, 70%, 80%, 90%, 95%, 99%, or 99.9%, compared to a standard, e.g., an expression level of the gene in e-ERYs in a normal subject. In some embodiments, the gene associated with GMPs is differentially expressed, e.g., up-regulated, by at least about 0.5, 1, 2, 5, 10, 20, 50, 100, 200, 500, or 1000 fold, compared to a standard, e.g., an expression level of the gene in GMPs in a normal subject. In some embodiments, the gene associated with DCs is differentially expressed, e.g., down-regulated, by at least about 10%, 20%, 30%, 40%, 50%, 60%, 70%, 80%, 90%, 95%, 99%, or 99.9%, or up-regulated, by at least about 0.5, 1, 2, 5, 10, 20, 50, 100, 200, 500, or 1000 fold, compared to a standard, e.g., an expression level of the gene in DCs in a normal subject. In some embodiments, the gene associated with NK cells is differentially expressed, e.g., down-regulated, by at least about 10%, 20%, 30%, 40%, 50%, 60%, 70%, 80%, 90%, 95%, 99%, or 99.9%, or up-regulated, by at least about 0.5, 1, 2, 5, 10, 20, 50, 100, 200, 500, or 1000 fold, compared to a standard, e.g., an expression level of the gene in NK cells in a normal subject.

In certain embodiments, the gene associated with B cells is a B cell-specific gene. In certain embodiments, the gene associated with T cells is a T cell-specific gene. In certain embodiments, the gene associated with e-ERYs is an e-ERY-specific gene. In certain embodiments, the gene associated with GMPs is a GMP-specific gene. In certain embodiments, the gene associated with DCs is a DC-specific gene. In certain embodiments, the gene associated with NK cells is a NK cell-specific gene.

In some embodiments, the method includes acquiring knowledge and/or evaluating a sample to determine if a subject has two or more (e.g., 2, 3, 4, 5, 6, 7, 8, 9, 10, 11, 12, 13, 14, 15, 16, 17, 18, 19, 20, 30, 40, 50, or more) genes, e.g., two or more genes described herein, differentially expressed (e.g., up-regulated or down-regulated), and, based upon that knowledge, administering the subject an MS therapy, e.g., an MS therapy described herein. In some embodiments, the two or more (e.g., 2, 3, 4, 5, 6, 7, 8, 9, 10, 11, 12, 13, 14, or 15) differentially expressed genes are selected from, e.g., FCRL1, IGHM, 231418_PM_at, CD22, IGH@, 217138_PM_x_at, POU2AF1, LOC283663, MS4A1, IGL@, TCL1A, MS4A1, IGHD, CLLU1, and IGK@. In some embodiments, the two or more genes are differentially expressed, e.g., down-regulated, by at least about 10%, 20%, 30%, 40%, 50%, 60%, 70%, 80%, 90%, 95%, 99%, or 99.9%, or up-regulated, by at least about 0.5, 1, 2, 5, 10, 20, 50, 100, 200, 500, or 1000 fold, compared to a standard, e.g., an expression level of the same gene in the same cell type in a normal subject.

In particular embodiments, the MS therapy comprises an anti-VLA-4 therapy, e.g., natalizumab. In particular embodiments, the MS therapy comprises an anti-CD25 therapy, e.g., daclizumab. In particular embodiments, the MS therapy comprises an interferon beta, e.g., interferon beta-1a or interferon beta-1b. In particular embodiments, the MS therapy comprises a sphingosine 1-phosphate (S1P) antagonist, e.g., fingolimod. In particular embodiments, the MS therapy comprises glatiramer acetate (GA). In certain embodiments, the subject has been treated with an MS therapy, e.g., an alternative MS therapy.

In a further aspect, the present invention provides a method of identifying a subject having multiple sclerosis (MS), e.g., secondary progressive multiple sclerosis (SPMS), or at risk of developing MS, e.g., SPMS, for treatment with an MS therapy, e.g., an MS therapy described herein.

In some embodiments, the method includes providing a sample, e.g., a blood sample, from a subject having multiple sclerosis (MS), e.g., secondary progressive multiple sclerosis (SPMS), or at risk of developing MS, e.g., SPMS, determining the expression levels of one or more (e.g., 1, 2, 3, 4, 5, or 6) of the following: two or more (e.g., 2, 3, 4, 5, 6, 7, 8, 9, 10, 25, 50, 100, 250, 500, or more) genes associated with B cells, two or more (e.g., 2, 3, 4, 5, 6, 7, 8, 9, 10, 25, 50, 100, 250, 500, or more) genes associated with T cells, two or more (e.g., 2, 3, 4, 5, 6, 7, 8, 9, 10, 25, 50, 100, 250, 500, or more) genes associated with e-ERYs, two or more (e.g., 2, 3, 4, 5, 6, 7, 8, 9, 10, 25, 50, 100, 250, 500, or more) genes associated with GMPs, two or more (e.g., 2, 3, 4, 5, 6, 7, 8, 9, 10, 25, 50, 100, 250, 500, or more) genes associated with DCs, and/or two or more (e.g., 2, 3, 4, 5, 6, 7, 8, 9, 10, 25, 50, 100, 250, 500, or more) genes associated with NK cells, in the sample; comparing the expression levels of the genes with a standard, e.g., an expression level of the same gene in the same cell type in a normal subject; and identifying the subject for treatment with an MS therapy, e.g., an MS therapy described herein, on the basis that the subject has one or more of the following: two or more genes associated with B cells differentially expressed (e.g., up-regulated), two or more genes associated with T cells differentially expressed (e.g., up-regulated), two or more genes associated with e-ERYs differentially expressed (e.g., up-regulated), two or more genes associated with GMPs differentially expressed (e.g., down-regulated), two or more genes associated with DCs differentially expressed (e.g., down-regulated or up-regulated), and/or two or more genes associated with NK cells differentially expressed (e.g., down-regulated or up-regulated).

In some embodiments, the method includes determining the expression levels of two or more (e.g., 2, 3, 4, 5, 6, 7, 8, 9, 10, 11, 12, 13, 14, 15, 16, 17, 18, 19, 20, 30, 40, 50, or more) genes described herein in the sample; comparing the expression levels of the genes with a standard, e.g., an expression level of the same gene in the same cell type in a normal subject; and identifying the subject for treatment with an MS therapy, e.g., an MS therapy described herein, on the basis that the subject has two or more of the genes differentially expressed (e.g., down-regulated or up-regulated). In some embodiments, the two or more (e.g., 2, 3, 4, 5, 6, 7, 8, 9, 10, 11, 12, 13, 14, or 15) differentially expressed genes are selected from, e.g., FCRL1, IGHM, 231418_PM_at, CD22, IGH@, 217138_PM_x_at, POU2AF1, LOC283663, MS4A1, IGL@, TCL1A, MS4A1, IGHD, CLLU1, and IGK@.

In some embodiments, the expression levels of the genes are determined by a method described herein, e.g., oligonucleotide array, an immunoassay (e.g., immunohistochemistry, northern blot, or a PCR method (e.g., quantitative RT-PCR). In some embodiments, expression levels of the genes are determined by evaluating the level of protein expression, e.g., by an immunoassay, e.g., by ELISA, immunohistochemistry, immunofluorescence, or western blot.

In certain embodiments, the expression levels are determined prior to initiating, during, or after, a treatment in the subject. In certain embodiments, the expression levels are determined at the time of diagnosis of the subject with MS, e.g., SPMS.

In some embodiments, the method includes comprising comparing the expression levels of the genes with a standard, e.g., an expression level of the same gene in the same cell type in a normal subject.

In some embodiments, the gene associated with B cells is differentially expressed, e.g., up-regulated, by at least about 0.5, 1, 2, 5, 10, 20, 50, 100, 200, 500, or 1000 fold, compared to a standard, e.g., an expression level of the gene in B cells in a normal subject. In some embodiments, the gene associated with T cells is differentially expressed, e.g., up-regulated, by at least about 0.5, 1, 2, 5, 10, 20, 50, 100, 200, 500, or 1000 fold, compared to a standard, e.g., an expression level of the gene in T cells in a normal subject. In some embodiments, the gene associated with e-ERYs is differentially expressed, e.g., up-regulated, by at least about 0.5, 1, 2, 5, 10, 20, 50, 100, 200, 500, or 1000 fold, compared to a standard, e.g., an expression level of the gene in e-ERYs in a normal subject. In some embodiments, the gene associated with GMPs is differentially expressed, e.g., down-regulated, by at least about 10%, 20%, 30%, 40%, 50%, 60%, 70%, 80%, 90%, 95%, or 99%, compared to a standard, e.g., an expression level of the gene in GMPs in a normal subject. In some embodiments, the gene associate with DCs is differentially expressed, e.g., down-regulated, by at least about 10%, 20%, 30%, 40%, 50%, 60%, 70%, 80%, 90%, 95%, or 99%, or up-regulated, by at least about 0.5, 1, 2, 5, 10, 20, 50, 100, 200, 500, or 1000 fold, compared to a standard, e.g., an expression level of the gene in DCs in a normal subject. In some embodiments, the gene associate with NK cells is differentially expressed, e.g., down-regulated, by at least about 10%, 20%, 30%, 40%, 50%, 60%, 70%, 80%, 90%, 95%, or 99%, or up-regulated, by at least about 0.5, 1, 2, 5, 10, 20, 50, 100, 200, 500, or 1000 fold, compared to a standard, e.g., an expression level of the gene in NK cells in a normal subject.

In certain embodiments, the gene associated with B cells is a B cell-specific gene. In certain embodiments, the gene associated with T cells is a T cell-specific gene. In certain embodiments, the gene associated with e-ERYs is an e-ERY-specific gene. In certain embodiments, the gene associated with GMPs is a GMP-specific gene. In certain embodiments, the gene associated with DCs is a DC-specific gene. In certain embodiments, the gene associated with NK cells is a NK cell-specific gene.

In some embodiments, the two or more (e.g., 2, 3, 4, 5, 6, 7, 8, 9, 10, 11, 12, 13, 14, 15, 16, 17, 18, 19, 20, 30, 40, 50, or more) gene described herein are differentially expressed, e.g., down-regulated, by at least about 10%, 20%, 30%, 40%, 50%, 60%, 70%, 80%, 90%, 95%, 99%, or 99.9%, or up-regulated, by at least about 0.5, 1, 2, 5, 10, 20, 50, 100, 200, 500, or 1000 fold, compared to a standard, e.g., an expression level of the same gene in the same cell type in a normal subject.

In particular embodiments, the MS therapy comprises an anti-VLA-4 therapy, e.g., natalizumab. In particular embodiments, the MS therapy comprises an anti-IL-2 receptor therapy, e.g., daclizumab. In particular embodiments, the MS therapy comprises an interferon beta, e.g., interferon beta-1a or interferon beta-1b. In particular embodiments, the MS therapy comprises a sphingosine 1-phosphate (SIP) antagonist, e.g., fingolimod. In particular embodiments, the MS therapy comprises glatiramer acetate (GA). In some embodiments, the subject has been treated with an MS therapy, e.g., an alternative MS therapy.

In another aspect, the present invention provides a method of treating or preventing one or more symptoms associated with MS, e.g., SPMS. The symptom can be a symptom described herein. In some embodiments, the method includes administering an MS therapy, e.g., an MS therapy described herein, to a subject. In some embodiments, the subject has MS, e.g., SPMS, or at risk of developing MS, e.g., SPMS, and the subject has one or more (e.g., 1, 2, 3, 4, 5, or 6) of the following: two or more (e.g., 2, 3, 4, 5, 6, 7, 8, 9, 10, 25, 50, 100, 250, 500, or more) genes associated with B cells differentially expressed (e.g., down-regulated), two or more (e.g., 2, 3, 4, 5, 6, 7, 8, 9, 10, 25, 50, 100, 250, 500, or more) genes associated with T cells differentially expressed (e.g., down-regulated), two or more (e.g., 2, 3, 4, 5, 6, 7, 8, 9, 10, 25, 50, 100, 250, 500, or more) genes associated with early erythrocytes (e-ERYs) differentially expressed (e.g., down-regulated), two or more (e.g., 2, 3, 4, 5, 6, 7, 8, 9, 10, 25, 50, 100, 250, 500, or more) genes associated with granulocyte/monocyte progenitors (GMPs) differentially expressed (e.g., up-regulated), two or more genes associated with DCs differentially expressed (e.g., down-regulated or up-regulated), and/or two or more genes associated with NK cells differentially expressed (e.g., down-regulated or up-regulated).

In some embodiments, the subject has MS, e.g., SPMS, or at risk of developing MS, e.g., SPMS, and the subject has two or more (e.g., 2, 3, 4, 5, 6, 7, 8, 9, 10, 11, 12, 13, 14, 15, 16, 17, 18, 19, 20, 30, 40, 50, or more) genes described herein differentially expressed (e.g., down-regulated or up-regulated) in the sample; comparing the expression levels of the genes with a standard, e.g., an expression level of the same gene in the same cell type in a normal subject; and identifying the subject for treatment with an MS therapy, e.g., an MS therapy described herein, on the basis that the subject has two or more of the genes differentially expressed (e.g., down-regulated or up-regulated). In some embodiments, the two or more (e.g., 2, 3, 4, 5, 6, 7, 8, 9, 10, 11, 12, 13, 14, or 15) differentially expressed genes are selected from, e.g., FCRL1, IGHM, 231418_PM_at, CD22, IGH@, 217138_PM_x_at, POU2AF1, LOC283663, MS4A1, IGL@, TCL1A, MS4A1, IGHD, CLLU1, and IGK@.

In yet another aspect, the present invention provides a method of treating or preventing one or more symptoms associated with MS, e.g., SPMS. The symptom can be a symptom described herein.

In some embodiments, the method includes administering an MS therapy, e.g., an MS therapy described herein, to a subject. In some embodiments, the subject has MS, e.g., SPMS, or at risk of developing MS, e.g., SPMS, and the subject has two or more (e.g., 2, 3, 4, 5, 6, 7, 8, 9, 10, 25, 50, 100, 250, 500, or more) genes associated with B cells differentially expressed (e.g., up-regulated), two or more (e.g., 2, 3, 4, 5, 6, 7, 8, 9, 10, 25, 50, 100, 250, 500, or more) genes associated with T cells differentially expressed (e.g., up-regulated), two or more (e.g., 2, 3, 4, 5, 6, 7, 8, 9, 10, 25, 50, 100, 250, 500, or more) genes associated with early erythrocytes (e-ERYs) differentially expressed (e.g., up-regulated), and two or more (e.g., 2, 3, 4, 5, 6, 7, 8, 9, 10, 25, 50, 100, 250, 500, or more) genes associated with granulocyte/monocyte progenitors (GMPs) differentially expressed (e.g., down-regulated), two or more (e.g., 2, 3, 4, 5, 6, 7, 8, 9, 10, 25, 50, 100, 250, 500, or more) genes associated with DCs differentially expressed (e.g., down-regulated or up-regulated), and/or two or more (e.g., 2, 3, 4, 5, 6, 7, 8, 9, 10, 25, 50, 100, 250, 500, or more) genes associated with NK cells differentially expressed (e.g., down-regulated or up-regulated).

In some embodiments, the subject has two or more (e.g., 2, 3, 4, 5, 6, 7, 8, 9, 10, 11, 12, 13, 14, 15, 16, 17, 18, 19, 20, 30, 40, 50, or more) genes described herein differentially expressed (e.g., down-regulated or up-regulated). In some embodiments, the two or more (e.g., 2, 3, 4, 5, 6, 7, 8, 9, 10, 11, 12, 13, 14, or 15) differentially expressed genes are selected from, e.g., FCRL1, IGHM, 231418_PM_at, CD22, IGH@, 217138_PM_x_at, POU2AF1, LOC283663, MS4A1, IGL@, TCL1A, MS4A1, IGHD, CLLU1, and IGK@.

In another aspect, the present invention provides a method of evaluating or monitoring clinical outcome, e.g., disease severity, disease progression, in a subject having multiple sclerosis (MS), e.g., secondary progressive multiple sclerosis (SPMS), or at risk of developing MS, e.g., SPMS.

In some embodiments, the method includes providing a sample, e.g., a blood sample, from a subject having multiple sclerosis (MS), e.g., secondary progressive multiple sclerosis (SPMS), or at risk of developing SPMS, determining the expression levels of one or more (e.g., 1, 2, 3, 4, 5, or 6) of the following: two or more (e.g., 2, 3, 4, 5, 6, 7, 8, 9, 10, 25, 50, 100, 250, 500, or more) genes associated with B cells, two or more (e.g., 2, 3, 4, 5, 6, 7, 8, 9, 10, 25, 50, 100, 250, 500, or more) genes associated with T cells, two or more (e.g., 2, 3, 4, 5, 6, 7, 8, 9, 10, 25, 50, 100, 250, 500, or more) genes associated with e-ERYs, two or more (e.g., 2, 3, 4, 5, 6, 7, 8, 9, 10, 25, 50, 100, 250, 500, or more) genes associated with GMPs, two or more (e.g., 2, 3, 4, 5, 6, 7, 8, 9, 10, 25, 50, 100, 250, 500, or more) genes associated with DCs, and/or two or more (e.g., 2, 3, 4, 5, 6, 7, 8, 9, 10, 25, 50, 100, 250, 500, or more) genes associated with NK cells, in the sample; comparing the expression levels of the genes with a standard, e.g., an expression level of the same gene in the same cell type in a normal subject; and evaluating or monitoring disease progression on the basis that the subject has one or more of the following: two or more genes associated with B cells differentially expressed (e.g., down-regulated), two or more genes associated with T cells differentially expressed (e.g., down-regulated), two or more genes associated with e-ERYs differentially expressed (e.g., down-regulated), two or more genes associated with GMPs differentially expressed (e.g., up-regulated), two or more genes associated with DCs differentially expressed (e.g., down-regulated or up-regulated), and/or two or more genes associated with NK cells differentially expressed (e.g., down-regulated or up-regulated).

In some embodiments, the method includes determining the expression levels of two or more (e.g., 2, 3, 4, 5, 6, 7, 8, 9, 10, 11, 12, 13, 14, 15, 16, 17, 18, 19, 20, 30, 40, 50, or more) genes described herein in the sample; comparing the expression levels of the genes with a standard, e.g., an expression level of the same gene in the same cell type in a normal subject; and evaluating or monitoring disease progression on the basis that the subject has two or more of the genes differentially expressed (e.g., down-regulated or up-regulated). In some embodiments, the two or more (e.g., 2, 3, 4, 5, 6, 7, 8, 9, 10, 11, 12, 13, 14, or 15) differentially expressed genes are selected from, e.g., FCRL1, IGHM, 231418_PM_at, CD22, IGH@, 217138_PM_x_at, POU2AF1, LOC283663, MS4A1, IGL@, TCL1A, MS4A1, IGHD, CLLU1, and IGK@.

In a further aspect, the present invention provides a method of evaluating or monitoring clinical outcome, e.g., disease severity, disease progression, in a subject having multiple sclerosis (MS), e.g., secondary progressive multiple sclerosis (SPMS), or at risk of developing SPMS. In some embodiments, the method includes providing a sample, e.g., a blood sample, from a subject having multiple sclerosis (MS), e.g., secondary progressive multiple sclerosis (SPMS), or at risk of developing MS, e.g., SPMS; determining the expression levels of one or more (e.g., 1, 2, 3, 4, 5, or 6) of the following: two or more genes associated with B cells, two or more genes associated with T cells, two or more genes associated with e-ERYs, two or more genes associated with GMPs, two or more genes associated with DCs, and/or two or more genes associated with NK cells, in the sample; comparing the expression levels of the genes with a standard, e.g., an expression level of the same gene in the same cell type in a normal subject; and identifying the subject on the basis that the subject has two or more genes associated with B cells differentially expressed (e.g., up-regulated), two or more genes associated with T cells differentially expressed (e.g., up-regulated), two or more genes associated with e-ERYs differentially expressed (e.g., up-regulated), two or more genes associated with GMPs down-regulated, two or more genes associated with DCs differentially expressed (e.g., down-regulated or up-regulated), and/or two or more genes associated with NK cells differentially expressed (e.g., down-regulated or up-regulated).

In some embodiments, the method includes determining the expression levels of two or more (e.g., 2, 3, 4, 5, 6, 7, 8, 9, 10, 11, 12, 13, 14, 15, 16, 17, 18, 19, 20, 30, 40, 50, or more) genes described herein in the sample; comparing the expression levels of the genes with a standard, e.g., an expression level of the same gene in the same cell type in a normal subject; and evaluating or monitoring disease progression on the basis that the subject has two or more of the genes differentially expressed (e.g., down-regulated or up-regulated). In some embodiments, the two or more (e.g., 2, 3, 4, 5, 6, 7, 8, 9, 10, 11, 12, 13, 14, or 15) differentially expressed genes are selected from, e.g., FCRL1, IGHM, 231418_PM_at, CD22, IGH@, 217138_PM_x_at, POU2AF1, LOC283663, MS4A1, IGL@, TCL1A, MS4A1, IGHD, CLLU1, and IGK@.

In yet another aspect, the present invention provides a method for generating a personalized MS, e.g., SPMS, treatment report, by obtaining a sample, e.g., a blood sample, from a subject having MS, e.g., SPMS, determining the expression levels of one or more (e.g., 1, 2, 3, 4, 5, or 6) of the following: two or more genes associated with B cells, two or more genes associated with T cells, two or more genes associated with e-ERYs, two or more genes associated with GMPs, two or more genes associated with DCs, and/or two or more genes associated with NK killers; and selecting an MS therapy, e.g., an MS therapy described herein, based on the expression levels identified, differential expression (e.g., down-regulation or up-regulation) of one or more (e.g., 1, 2, 3, 4, 5, or 6) of the following: differential expression (e.g., down-regulation) of two or more (e.g., 2, 3, 4, 5, 6, 7, 8, 9, 10, 25, 50, 100, 250, 500, or more) genes associated with B cells, differential expression (e.g., down-regulation) of two or more (e.g., 2, 3, 4, 5, 6, 7, 8, 9, 10, 25, 50, 100, 250, 500, or more) genes associated with T cells, differential expression (e.g., down-regulation) of two or more (e.g., 2, 3, 4, 5, 6, 7, 8, 9, 10, 25, 50, 100, 250, 500, or more) genes associated with early erythrocytes (e-ERYs), differential expression (e.g., up-regulation) of two or more (e.g., 2, 3, 4, 5, 6, 7, 8, 9, 10, 25, 50, 100, 250, 500, or more) genes associated with granulocyte/monocyte progenitors (GMPs), differential expression (e.g., down-regulation or up-regulation) of two or more (e.g., 2, 3, 4, 5, 6, 7, 8, 9, 10, 25, 50, 100, 250, 500, or more) genes associated with DCs, and/or differential expression (e.g., down-regulation or up-regulation) of two or more (e.g., 2, 3, 4, 5, 6, 7, 8, 9, 10, 25, 50, 100, 250, 500, or more) genes associated with NK cells, indicates a first course of treatment; and differential expression (e.g., up-regulation or down-regulation) of one or more (e.g., 1, 2, 3, 4, 5, or 6) of the following: differential expression (e.g., up-regulation) of two or more (e.g., 2, 3, 4, 5, 6, 7, 8, 9, 10, 25, 50, 100, 250, 500, or more) genes associated with B cells, differential expression (e.g., up-regulation) of two or more (e.g., 2, 3, 4, 5, 6, 7, 8, 9, 10, 25, 50, 100, 250, 500, or more) genes associated with T cells, differential expression (e.g., up-regulation) of two or more (e.g., 2, 3, 4, 5, 6, 7, 8, 9, 10, 25, 50, 100, 250, 500, or more) genes associated with early erythrocytes (e-ERYs), differential expression (e.g., down-regulation) of two or more (e.g., 2, 3, 4, 5, 6, 7, 8, 9, 10, 25, 50, 100, 250, 500, or more) genes associated with granulocyte/monocyte progenitors (GMPs), differential expression (e.g., down-regulation or up-regulation) of two or more (e.g., 2, 3, 4, 5, 6, 7, 8, 9, 10, 25, 50, 100, 250, 500, or more) genes associated with DCs, and/or differential expression (e.g., down-regulation or up-regulation) of two or more (e.g., 2, 3, 4, 5, 6, 7, 8, 9, 10, 25, 50, 100, 250, 500, or more) genes associated with NK cells, indicates a second different course of treatment.

In some embodiments, the method includes determining the expression levels of two or more (e.g., 2, 3, 4, 5, 6, 7, 8, 9, 10, 11, 12, 13, 14, 15, 16, 17, 18, 19, 20, 30, 40, 50, or more) genes described herein in the sample; selecting an MS therapy, e.g., an MS therapy described herein, based on the expression levels identified. In some embodiments, differential expression (e.g., up-regulation) of two or more of the genes indicates a first course of treatment. In some embodiments, differential expression (e.g., down-regulation) of two or more of the genes indicates a second different course of treatment. In some embodiments, the two or more (e.g., 2, 3, 4, 5, 6, 7, 8, 9, 10, 11, 12, 13, 14, or 15) differentially expressed genes are selected from, e.g., FCRL1, IGHM, 231418_PM_at, CD22, IGH@, 217138_PM_x_at, POU2AF1, LOC283663, MS4A1, IGL@, TCL1A, MS4A1, IGHD, CLLU1, and IGK@.

In some embodiments, the first course of treatment comprises an MS therapy described herein. In some embodiments, the second course of treatment comprises a different MS therapy described herein.

In another aspect, the present invention provides a method of determining a gene expression profile for a subject having MS, e.g., SPMS. In some embodiments, the method includes directly acquiring knowledge of the expression levels of one or more (e.g., 1, 2, 3, 4, 5, or 6) of the following: two or more (e.g., 2, 3, 4, 5, 6, 7, 8, 9, 10, 25, 50, 100, 250, 500, or more) genes associated with B cells, two or more (e.g., 2, 3, 4, 5, 6, 7, 8, 9, 10, 25, 50, 100, 250, 500, or more) genes associated with T cells, two or more (e.g., 2, 3, 4, 5, 6, 7, 8, 9, 10, 25, 50, 100, 250, 500, or more) genes associated with e-ERYs, two or more (e.g., 2, 3, 4, 5, 6, 7, 8, 9, 10, 25, 50, 100, 250, 500, or more) genes associated with GMPs, two or more (e.g., 2, 3, 4, 5, 6, 7, 8, 9, 10, 25, 50, 100, 250, 500, or more) genes associated with DCs, and/or two or more (e.g., 2, 3, 4, 5, 6, 7, 8, 9, 10, 25, 50, 100, 250, 500, or more) genes associated with NK cells, in a sample from a subject having MS, e.g., SPMS, and responsive to a determination of differential expression (e.g., down-regulation or up-regulation) of the genes, one or more of: (1) stratifying a subject population; (2) identifying or selecting the subject as likely or unlikely to respond to an MS therapy, e.g., an MS therapy described herein; (3) selecting an MS therapy, e.g., an MS therapy described herein; (4) treating the subject with an MS therapy, e.g., an MS therapy described herein; or (5) prognosticating the time course and/or severity of the disease in the subject.

In some embodiments, the method includes directly acquiring knowledge of the expression levels of two or more (e.g., 2, 3, 4, 5, 6, 7, 8, 9, 10, 11, 12, 13, 14, 15, 16, 17, 18, 19, 20, 30, 40, 50, or more) genes described herein, and, based upon that knowledge, administering the subject an MS therapy, e.g., an MS therapy described herein. In some embodiments, the two or more (e.g., 2, 3, 4, 5, 6, 7, 8, 9, 10, 11, 12, 13, 14, or 15) genes are selected from, e.g., FCRL1, IGHM, 231418_PM_at, CD22, IGH@, 217138_PM_x_at, POU2AF1, LOC283663, MS4A1, IGL@, TCL1A, MS4A1, IGHD, CLLU1, and IGK@.

In some embodiments, responsive to the direct acquisition of knowledge of the expression levels of the genes, the subject is classified as a candidate to receive an MS therapy, e.g., an MS therapy described herein. In some embodiments, responsive to the direct acquisition of knowledge of the expression levels of the genes, the subject is identified as likely to respond to an MS therapy, e.g., an MS therapy described herein.

In a further aspect, the present invention provides a reaction mixture including: a plurality of detection reagents, or purified or isolated preparation thereof; and a target nucleic acid preparation derived from a sample, e.g., a blood sample, from a subject having MS, e.g., SPMS. In some embodiments, the plurality of detection reagents can determine expression levels of one or more (e.g., 1, 2, 3, 4, 5, or 6) of the following: two or more (e.g., 2, 3, 4, 5, 6, 7, 8, 9, 10, 25, 50, 100, 250, 500, or more) genes associated with B cells, two or more (e.g., 2, 3, 4, 5, 6, 7, 8, 9, 10, 25, 50, 100, 250, 500, or more) genes associated with T cells, two or more (e.g., 2, 3, 4, 5, 6, 7, 8, 9, 10, 25, 50, 100, 250, 500, or more) genes associated with e-ERYs, two or more (e.g., 2, 3, 4, 5, 6, 7, 8, 9, 10, 25, 50, 100, 250, 500, or more) genes associated with GMPs, two or more (e.g., 2, 3, 4, 5, 6, 7, 8, 9, 10, 25, 50, 100, 250, 500, or more) genes associated with DC cells, and/or two or more (e.g., 2, 3, 4, 5, 6, 7, 8, 9, 10, 25, 50, 100, 250, 500, or more) genes associated with NK cells.

In some embodiments, the plurality of detection reagents can determine the expression levels of two or more (e.g., 2, 3, 4, 5, 6, 7, 8, 9, 10, 11, 12, 13, 14, 15, 16, 17, 18, 19, 20, 30, 40, 50, or more) genes described herein in the sample. In some embodiments, the two or more (e.g., 2, 3, 4, 5, 6, 7, 8, 9, 10, 11, 12, 13, 14, or 15) genes are selected from, e.g., FCRL1, IGHM, 231418_PM_at, CD22, IGH@, 217138_PM_x_at, POU2AF1, LOC283663, MS4A1, IGL@, TCL1A, MS4A1, IGHD, CLLU1, and IGK@. The detection reagent can comprise a probe to measure the expression level of the gene.

In another aspect, the invention provides methods of making a reaction mixture comprising combining a plurality of detection reagents, with a target nucleic acid preparation comprising plurality of target nucleic acid molecules derived from a sample, e.g., from a blood sample, from a subject having MS, e.g., SPMS.

In some embodiments, the plurality of detection reagents can determine expression levels of one or more (1, 2, 3, 4, 5, or 6) of the following: two or more (e.g., 2, 3, 4, 5, 6, 7, 8, 9, 10, 25, 50, 100, 250, 500, or more) genes associated with B cells, two or more (e.g., 2, 3, 4, 5, 6, 7, 8, 9, 10, 25, 50, 100, 250, 500, or more) genes associated with T cells, two or more (e.g., 2, 3, 4, 5, 6, 7, 8, 9, 10, 25, 50, 100, 250, 500, or more) genes associated with e-ERYs, two or more (e.g., 2, 3, 4, 5, 6, 7, 8, 9, 10, 25, 50, 100, 250, 500, or more) genes associated with GMPs, two or more (e.g., 2, 3, 4, 5, 6, 7, 8, 9, 10, 25, 50, 100, 250, 500, or more) genes associated with DCs, and/or two or more (e.g., 2, 3, 4, 5, 6, 7, 8, 9, 10, 25, 50, 100, 250, 500, or more) genes associated with NK cells.

In some embodiments, the plurality of detection reagents can determine expression levels of two or more (e.g., 2, 3, 4, 5, 6, 7, 8, 9, 10, 11, 12, 13, 14, 15, 16, 17, 18, 19, 20, 30, 40, 50, or more) genes described herein. In some embodiments, the two or more (e.g., 2, 3, 4, 5, 6, 7, 8, 9, 10, 11, 12, 13, 14, or 15) differentially expressed genes are selected from, e.g., FCRL1, IGHM, 231418_PM_at, CD22, IGH@, 217138_PM_x_at, POU2AF1, LOC283663, MS4A1, IGL@, TCL1A, MS4A1, IGHD, CLLU1, and IGK@. The detection reagent can comprise a probe to measure the expression level of the gene.

In another aspect, the present invention provides a system for evaluating a subject population having MS, e.g., SPMS, the system comprising at least one processor operatively connected to a memory, the at least one processor has: a first plurality of values for a plurality of subjects having MS, e.g., SPMS, wherein each value is indicative of expression of a gene, e.g., a gene associated with T cells, B cells, e-ERYs, GMPs, DCs, and/or NK cells; a second plurality of values for the plurality of subjects having MS, e.g., SPMS, wherein each value is indicative of a clinical score for a subject having MS, e.g., SPMS, e.g., a clinical score associated with disease severity, disease progression, clinical outcome, or prognosis, e.g., a clinical score described herein, e.g., Expanded Disability Status Scale (EDSS), or Multiple Sclerosis Severity Score (MSSS); and a function that correlates the first plurality of values with the second plurality of values to provide an output of classification of the MS, e.g., SPMS, of the subject population.

In some embodiments, the correlative function determines the joint distribution of the plurality of the subjects in a space of gene expression and clinical score (e.g., a clinical score described herein), e.g., by a method described herein, e.g., using one or more steps described in FIG. 6. In certain embodiments, the correlative function determines the joint distribution of the plurality of the subjects in a space of gene expression (X) and clinical score (Y), e.g., by the likelihood maximization problem:

$$Θ = {argmax}_{Θ} P ( Y , X )$$

$$P ( Y , X ) = {\sum }_{m = 1}^{K} P ( Y , X , {c}_{m} ) = {\sum }_{m = 1}^{K} P ( Y \vert X , {c}_{m} ) P ( X \vert {c}_{m} ) P ( {c}_{m} )$$

where Θ represents the set of parameters used to describe the joint distribution, which includes parameters for the linear regression used to describe P(Y|X,cₘ), parameters for describing the clusters P(X|cₘ) and P(cₘ). In some embodiments, the correlative function uses a regularized Expectation-Maximization algorithm (EM) to learn a sparse set of parameters. In some embodiments, the output indicates an optimal number of clusters for the subject population, e.g., using Bayesian information criterion (BIC).

In yet another aspect, the present invention provides a kit for identifying a subject having multiple sclerosis (MS), e.g., secondary progressive multiple sclerosis (SPMS), or at risk of developing MS, e.g., SPMS, for treatment with an MS therapy, e.g., an MS therapy described herein, and/or for identifying a clinical outcome (e.g., disease severity or disease progression) for a subject having MS, e.g., SPMS.

In some embodiments, the kit includes a product comprising a plurality of agents capable of interacting with a gene expression product of a plurality of genes, wherein the agents detect the expression levels of one or more (e.g., 1, 2, 3, 4, 5, or 6) of the following: two or more (e.g., 2, 3, 4, 5, 6, 7, 8, 9, 10, 25, 50, 100, 250, 500, or more) genes associated with B cells, two or more (e.g., 2, 3, 4, 5, 6, 7, 8, 9, 10, 25, 50, 100, 250, 500, or more) genes associated with T cells, two or more (e.g., 2, 3, 4, 5, 6, 7, 8, 9, 10, 25, 50, 100, 250, 500, or more) genes associated with e-ERYs, two or more (e.g., 2, 3, 4, 5, 6, 7, 8, 9, 10, 25, 50, 100, 250, 500, or more) genes associated with GMPs, two or more (e.g., 2, 3, 4, 5, 6, 7, 8, 9, 10, 25, 50, 100, 250, 500, or more) genes associated with DCs, and/or two or more (e.g., 2, 3, 4, 5, 6, 7, 8, 9, 10, 25, 50, 100, 250, 500, or more) genes associated with NK cells, in a sample.

In some embodiments, the agents detect the expression levels of two or more (e.g., 2, 3, 4, 5, 6, 7, 8, 9, 10, 11, 12, 13, 14, 15, 16, 17, 18, 19, 20, 30, 40, 50, or more) genes described herein in a sample. In some embodiments, the two or more (e.g., 2, 3, 4, 5, 6, 7, 8, 9, 10, 11, 12, 13, 14, or 15) genes are selected from, e.g., FCRL1, IGHM, 231418_PM_at, CD22, IGH@, 217138_PM_x_at, POU2AF1, LOC283663, MS4A1, IGL@, TCL1A, MS4A1, IGHD, CLLU1, and IGK@.

In another aspect, the present invention provides an in vitro method of determining if a subject having MS, e.g., SPMS, is a potential candidate for an MS therapy, e.g., an MS therapy described herein. The method comprises determining the expression levels of one or more (e.g., 1, 2, 3, 4, 5, or 6) of the following: two or more (e.g., 2, 3, 4, 5, 6, 7, 8, 9, 10, 25, 50, 100, 250, 500, or more) genes associated with B cells, two or more (e.g., 2, 3, 4, 5, 6, 7, 8, 9, 10, 25, 50, 100, 250, 500, or more) genes associated with T cells, two or more (e.g., 2, 3, 4, 5, 6, 7, 8, 9, 10, 25, 50, 100, 250, 500, or more) genes associated with e-ERYs, two or more (e.g., 2, 3, 4, 5, 6, 7, 8, 9, 10, 25, 50, 100, 250, 500, or more) genes associated with GMPs, two or more (e.g., 2, 3, 4, 5, 6, 7, 8, 9, 10, 25, 50, 100, 250, 500, or more) genes associated with DCs, and/or two or more (e.g., 2, 3, 4, 5, 6, 7, 8, 9, 10, 25, 50, 100, 250, 500, or more) genes associated with NK cells.

In some embodiments, the method includes determining the expression levels of two or more (e.g., 2, 3, 4, 5, 6, 7, 8, 9, 10, 11, 12, 13, 14, 15, 16, 17, 18, 19, 20, 30, 40, 50, or more) genes described herein. In some embodiments, the two or more (e.g., 2, 3, 4, 5, 6, 7, 8, 9, 10, 11, 12, 13, 14, or 15) genes are selected from, e.g., FCRL1, IGHM, 231418_PM_at, CD22, IGH@, 217138_PM_x_at, POU2AF1, LOC283663, MS4A1, IGL@, TCL1A, MS4A1, IGHD, CLLU1, and IGK@.

In some embodiments, optionally, the method further includes treating the subject with an MS therapy, e.g., an MS therapy described herein, or withholding treatment to the subject of an MS therapy, e.g., an MS therapy described herein.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 depicts an exemplary traditional paradigm of patient stratification. Assumption is that distinct molecular subgroups correspond to different disease severity distributions.

FIG. 2 depicts a mixture of experts toy model. (Left) Univariate case: Y-axis represents the clinical measurement, and X-axis represents the molecular profiles. The colors indicate different dependence relations between Y and X. (Right) Multivariate case: Two groups are determined by the differential expression of g1, g2 and g3 (blue=low and red=high). But the genes g4 and g5, that do not show the same level of differential expression, correlate with the clinical score.

FIG. 3 depicts an exemplary non-negative matrix factorization (NMF) which was used to reduce the dimensionality of the molecular profiles. Number of reduced dimensions (factors) was chosen by maximizing cophenetic correlation.

FIG. 4 depicts an exemplary map of the factors to different cell types using cell-specific expression pattern from D-MAP data. The most differentiating cell types in the two clusters were: T cell, B cell, e-Erythrocyte cells.

FIG. 5 depicts exemplary different dependence between molecular factors and disease severity in the two sub-groups.

FIG. 6 depicts exemplary steps in mixture of experts model for patient stratification.

FIG. 7 depicts exemplary top genes differentially expressed between the SPMS subgroups.

FIG. 8 depicts exemplary different dependence between molecular factors and disease severity in the SPMS subgroups.

FIG. 9 depicts BIC for 1-3 subgroups of SPMS samples. Median BIC for 3 subgroups is lower then 2, however with this data set the 3 subgroups led to highly variable BIC. Thus, the model mixture of 2 experts was selected.

FIG. 10 depicts density plots for the MSSS distribution in the two subgroups. The peak of subgroup A is shifted towards the right.

FIG. 11 depicts the drug usage in ACP MSSS based clustering, SPMS_{A} (right) and SPMS_{B} (left).

### DETAILED DESCRIPTION

The invention is based, at least in part, on the discovery that subgroups of MS, e.g., SPMS, patients can be identified by characterizing high or low expression of cell markers specific for, for example, B cells, T cells, and early erythrocyte cells, and that within each subgroup a different molecular signature can reflect a disease score.

### DEFINITIONS

As used herein, the term “acquire” or “acquiring” refers to obtaining possession of a physical entity, or a value, e.g., a numerical value, by “directly acquiring” or “indirectly acquiring” the physical entity or the value. “Directly acquiring” means performing a process (e.g., performing an assay or test on a sample or “analyzing a sample” as that term is defined herein) to obtain the physical entity or value. “Indirectly acquiring” refers to receiving the physical entity or value from another party or source (e.g., a third party laboratory that directly acquired the physical entity or value). Directly acquiring a physical entity includes performing a process, e.g., analyzing a sample, that includes a physical change in a physical substance, e.g., a starting material. Exemplary changes include making a physical entity from two or more starting materials, shearing or fragmenting a substance, separating or purifying a substance, combining two or more separate entities into a mixture, performing a chemical reaction that includes breaking or forming a covalent or non-covalent bond. Directly acquiring a value includes performing a process that includes a physical change in a sample or another substance, e.g., performing an analytical process which includes a physical change in a substance, e.g., a sample, analyte, or reagent (sometimes referred to herein as “physical analysis”), performing an analytical method, e.g., a method which includes one or more of the following: separating or purifying a substance, e.g., an analyte, or a fragment or other derivative thereof, from another substance; combining an analyte, or fragment or other derivative thereof, with another substance, e.g., a buffer, solvent, or reactant; or changing the structure of an analyte, or a fragment or other derivative thereof, e.g., by breaking or forming a covalent or non-covalent bond, between a first and a second atom of the analyte; or by changing the structure of a reagent, or a fragment or other derivative thereof, e.g., by breaking or forming a covalent or non-covalent bond, between a first and a second atom of the reagent.

As used herein, “analyzing” a sample includes performing a process that involves a physical change in a sample or another substance, e.g., a starting material. Exemplary changes include making a physical entity from two or more starting materials, shearing or fragmenting a substance, separating or purifying a substance, combining two or more separate entities into a mixture, performing a chemical reaction that includes breaking or forming a covalent or non-covalent bond. Analyzing a sample can include performing an analytical process which includes a physical change in a substance, e.g., a sample, analyte, or reagent (sometimes referred to herein as “physical analysis”), performing an analytical method, e.g., a method which includes one or more of the following: separating or purifying a substance, e.g., an analyte, or a fragment or other derivative thereof, from another substance; combining an analyte, or fragment or other derivative thereof, with another substance, e.g., a buffer, solvent, or reactant; or changing the structure of an analyte, or a fragment or other derivative thereof, e.g., by breaking or forming a covalent or non-covalent bond, between a first and a second atom of the analyte; or by changing the structure of a reagent, or a fragment or other derivative thereof, e.g., by breaking or forming a covalent or non-covalent bond, between a first and a second atom of the reagent.

### Subjects

Patients having MS may be identified by criteria establishing a diagnosis of clinically definite MS as defined by the workshop on the diagnosis of MS (Poser et al., Ann. Neurol. 13:227, 1983). Briefly, an individual with clinically definite MS has had two attacks and clinical evidence of either two lesions or clinical evidence of one lesion and paraclinical evidence of another, separate lesion. Definite MS may also be diagnosed by evidence of two attacks and oligoclonal bands of IgG in cerebrospinal fluid or by combination of an attack, clinical evidence of two lesions and oligoclonal band of IgG in cerebrospinal fluid. The McDonald criteria can also be used to diagnose MS. (McDonald et al., 2001, Recommended diagnostic criteria for multiple sclerosis: guidelines from the International Panel on the Diagnosis of Multiple Sclerosis, Ann Neurol 50:121-127). The McDonald criteria include the use of MRI evidence of CNS impairment over time to be used in diagnosis of MS, in the absence of multiple clinical attacks.

MS may be evaluated in several different ways. Exemplary criteria include: EDSS (Expanded Disability Status Scale), MSSS (Multiple Sclerosis Severity Score), KPS (Karnofsky Performance Scale, and appearance of exacerbations on MRI (Magnetic Resonance Imaging). The EDSS is a means to grade clinical impairment due to MS (Kurtzke, Neurology 33:1444, 1983). Eight functional systems are evaluated for the type and severity of neurologic impairment. Briefly, patients are evaluated for impairment in the following systems: pyramidal, cerebella, brainstem, sensory, bowel and bladder, visual, cerebral, and other. Follow-ups are conducted at defined intervals. The scale ranges from 0 (normal) to 10 (death due to MS). In evaluating effectiveness of MS treatment, a decrease of one full step indicates an effective treatment (Kurtzke, Ann. Neurol. 36:573-79, 1994).

TABLE 1
Expanded Disability Status Scale (Kurtzke JF.
Neurology. 1983; 33: 1444-1452)

| EDSS | Description |
|---|---|
| 0.0 | Normal neurological exam |
| 1.0 | No disability, minimal signs on 1 FS |
| 1.5 | No disability, minimal signs on 2 of 7 FS |
| 2.0 | Minimal disability in 1 of 7 FS |
| 2.5 | Minimal disability in 2 FS |
| 3.0 | Moderate disability in 1 FS; or mild disability in 3-4 FS, |
| | though fully ambulatory |
| 3.5 | Fully ambulatory but with moderate disability in 1 FS; |
| | mild disability in 1 or 2 FS; moderate |

disability in 2 FS; or mild disability in 5 FS

| | |
|---|---|
| 4.0 | Fully ambulatory without aid, up and about 12 |
| | hours a day despite relatively severe |
| | disability; able to walk without aid 500 meters |
| 4.5 | Fully ambulatory without aid; up and about much |
| | of the day; able to work a full day; may |
| | otherwise have some limitations of full activity |
| | or require minimal assistance; relatively |

severe disability; able to walk without aid 300 meters

| | |
|---|---|
| 5.0 | Ambulatory without aid for about 200 meters; |
| | disability impairs full daily activities |
| 5.5 | Ambulatory for 100 meters; disability precludes |
| | full daily activities |
| 6.0 | Intermittent or unilateral constant assistance |
| | (cane, crutch, or brace) required to walk |
| | 100 meters with or without resting |
| 6.5 | Constant bilateral support (cane, crutch, or braces) |
| | required to walk 20 meters without resting |
| 7.0 | Unable to walk beyond 5 meters even with aid, |
| | essentially restricted to wheelchair, wheels self, |
| | transfers alone; active in wheelchair about 12 hours a day |
| 7.5 | Unable to take more than a few steps; |
| | restricted to wheelchair; may need aid to transfer; |
| | wheels self, but may require motorized chair for full day |
| 8.0 | Essentially restricted to bed, chair, or wheelchair, |
| | but may be out of bed much of day; retains |
| | self-care functions; generally effective use of arms |
| 8.5 | Essentially restricted to bed much of day; some effective |
| | use of arms; retains some self-care functions |
| 9.0 | Helpless bed patient; can communicate and eat |
| 9.5 | Unable to communicate effectively or eat/swallow |
| 10.0 | Death due to MS |

FS = functional system(s).

The MSSS is an algorithm that relates scores on the EDSS to the distribution of disability in patients with comparable disease durations (Roxburgh, et al. Neurology 64:1144, 2005). Thus, similar relatively high MSSS numbers will be assigned to patients who accrue moderate disability over a short period of time, or severe disability over a moderate period of time (Pachner, et al. J Neurol Sci 278(1-2): 66, 2009). The MSSS is a powerful method for comparing disease progression using single assessment data, and can be used as a reference table for future disability comparisons (Roxburgh, et al. Neurology 64:1144, 2005).

TABLE 2
The Karnofsky Performance Scale (Karnofsky D, Burchenal J.
Evaluation of Chemotherapeutic Agents. New York, NY: Columbia
University Press; 1949)
Karnofsky Performance Scale

| | Percentage | |
|---|---|---|
| Progression | (%) | Description |
| Mild | 100 | Normal; no complaints; no |
| Able to carry on normal | | evidence of disease |
| activity and to work; no | 90 | Able to carry on normal |
| special care needed | | activity; minor signs or |
| | | symptoms of disease |
| | 80 | Normal activity with effort; |
| | | some signs or symptoms of |
| | | disease |
| Moderate | 70 | Cares for self; unable to carry |
| Unable to work; able to live at | | on normal activity or do active |
| home and care for most | | work |
| personal needs; varying | 60 | Requires occasional |
| amount of assistance needed | | assistance; able to care for |
| | | most personal needs |
| | 50 | Requires considerable |
| | | assistance and frequent |
| | | medical care |
| Severe | 40 | Disabled; requires special care |
| Unable to care for self; | | and assistance |
| requires equivalent of | 30 | Severely disabled; hospital |
| institutional or hospital care; | | admission is indicated; death |
| disease may be progressing | | not imminent |
| rapidly | 20 | Very sick; hospital admission |
| | | necessary; active supportive |
| | | treatment necessary |
| | 10 | Moribund; fatal processes |
| | | progressing rapidly |
| | 0 | Death |

Exacerbations on MRI are defined as the appearance of a new symptom that is attributable to MS and accompanied by an appropriate new neurologic abnormality (IFNB MS Study Group, supra). In addition, the exacerbation must last at least 24 hours and be preceded by stability or improvement for at least 30 days. Briefly, patients are given a standard neurological examination by clinicians. Exacerbations are mild, moderate, or severe according to changes in a Neurological Rating Scale (Sipe et al., Neurology 34:1368, 1984). An annual exacerbation rate and proportion of exacerbation-free patients are determined.

Standard Subject:

As used herein, the term “standard subject” or “control subject” refers to a subject who has standard or control level of disease, e.g., multiple sclerosis. In some cases, such a standard or control subject is a “normal subject,” e.g., a healthy subject, e.g., a subject who has not been diagnosed with MS, a subject who currently shows no signs of MS, a subject who has not previously shown signs of MS. In some cases, such standard or control subjects have low levels of disease, e.g., non-clinically definite MS (e.g., clinically isolated syndrome (CIS)), low severity MS (e.g., low EDSS score, low MSSS score), non-progressive MS (e.g., relapsing remitting MS (RRMS)), primary progressive MS (PPMS), or recently developed secondary progressive MS.

### Molecular Patient Stratification

High-throughput profiling technologies, e.g., genetic, transcriptomic, and proteomic approaches, can provide molecular profiles of patient samples. The goal of analyzing molecular profiles is, e.g., to understand to what extent the clinical variability can be explained by the molecular variability. Biomarkers associated with clinical features provide insights into molecular mechanisms underlying the disease and thus contribute to the selection of targeted therapies.

Methods that can be used for molecular patient stratification include, e.g., traditional approaches that investigate the molecular profiles independently of the clinical score (Cancer Genome Atlas Network, Nature 490(7418): 61-70, 2012; Chaussabel et al. Immunity 29(1):150-164, 2008; Ottoboni et al. Sci Transl Med 4(153):153ra131, 2012; Perou et al. Proc Natl Acad Sci USA 96(16): 9212-9217, 1999). An initial dimensionality reduction can be used to search for markers associating with disease severity score in the entire cohort and then for subgroups defined by these markers (Wang et al. Lancet 365(9460): 671-679, 2005). After an initial step of dimensionality reduction, an unsupervised clustering can be performed to identify molecularly uniform subgroups of samples. If such sub-groups are identified, next disease or progression scores can be associated with these.

Traditional approaches assume that molecular subgroups will reflect variability in disease severity and/or progression classes. Some methods add biologically motivated constraints to the clustering to aid in interpretation. For example, NBS (Network Based Stratification) (Hofree et al. Nat Methods 10(11): 1108-1115, 2013) algorithm identifies patient subgroups that show similar network characteristics and mutational profiles. Other methods analyze molecular markers differentiating pre-defined patient or healthy control groups, e.g., investigating whole-blood RNA transcripts differentiating MS patients from healthy controls (Nickles et al. Hum Mol Genet 22(20): 4194-4205, 2013).

Traditional approaches identify molecular sub-groups first and subsequent analyzes disease progression determined that there are indeed significant differences between the groups. The approach described herein simultaneously discovers molecular subclasses of patients' samples and molecular features that explain the clinical variability. The rationale is, e.g., that molecularly uniform patient samples represents a more uniform disease severity, prognosis or drug response. The method described herein indicates that in the joint space of molecular and disease scores, there exists distinct subgroups of patients such that each group is characterized by a different dependence between molecular and disease scores. This approach finds molecular characteristics defining uniform sample subsets and possibly an independent set of characteristics that explain the clinical variability. This approach does not implicitly enforce the constraint that variables defining molecularly distinct subtypes also explain the clinical variability.

### Treatment and MS Therapies

The methods described herein can be used to treat a subject having MS, e.g., SPMS, or at risk of developing MS, e.g., SPMS, or to treat or prevent a symptom associated with MS, e.g., SPMS.

As used herein, the term “treating” refers to partially or completely alleviating, ameliorating, improving, relieving, delaying onset of, inhibiting progression of, reducing severity of, and/or reducing incidence of one or more symptoms, features, or clinical manifestations of a particular disease, disorder, and/or condition. Treatment may be administered to a subject who does not exhibit signs of a disease, disorder, and/or condition (e.g., prior to an identifiable symptom) and/or to a subject who exhibits only early signs of a disease, disorder, and/or condition for the purpose of decreasing the risk of developing pathology associated with the disease, disorder, and/or condition.

As used herein, the term “preventing” refers to partially or completely delaying onset of MS, e.g., SPMS; partially or completely delaying onset of one or more symptoms, features, or clinical manifestations of a particular disease, disorder, and/or condition associated with MS, SPMS; partially or completely delaying onset of one or more symptoms, features, or manifestations of a particular disease, disorder, and/or condition prior to an identifiable symptom; partially or completely delaying progression from an latent disease, disorder and/or condition to an active disease, disorder and/or condition; and/or decreasing the risk of developing pathology associated with the disease, disorder, and/or condition.

In certain embodiments, the MS therapy is an anti-VLA-4 therapy. An anti-VLA-4 therapy is a molecule, e.g., a small molecule compound or protein biologic (e.g., an antibody or fragment thereof, such as an antigen-binding fragment thereof) that blocks VLA-4 activity. The molecule that is the anti-VLA-4 therapy is a VLA-4 antagonist. A VLA-4 antagonist includes any compound that inhibits a VLA-4 integrin from binding a ligand and/or receptor. An anti-VLA-4 therapy can be an antibody (e.g., natalizumab (TYSABRI®)) or fragment thereof, or a soluble form of a ligand. Soluble forms of the ligand proteins for α4 integrins include soluble VCAM-I or fibronectin peptides, VCAM-I fusion proteins, or bifunctional VCAM-I/Ig fusion proteins. For example, a soluble form of a VLA-4 ligand or a fragment thereof may be administered to bind to VLA-4, and in some instances, compete for a VLA-4 binding site on cells, thereby leading to effects similar to the administration of antagonists such as anti-VLA-4 antibodies. For example, soluble VLA-4 integrin mutants that bind VLA-4 ligand but do not elicit integrin-dependent signaling are suitable for use in the described methods. Such mutants can act as competitive inhibitors of wild type integrin protein and are considered “antagonists.” Other suitable antagonists are “small molecules.”

“Small molecules” are agents that mimic the action of peptides to disrupt VLA-4/ligand interactions by, for instance, binding VLA-4 and blocking interaction with a VLA-4 ligand (e.g., VCAM-I or fibronectin), or by binding a VLA-4 ligand and preventing the ligand from interacting with VLA-4. One exemplary small molecule is an oligosaccharide that mimics the binding domain of a VLA-4 ligand (e.g., fibronectin or VCAM-I) and binds the ligand-binding domain of VLA-4. (See, Devlin et al., Science 249: 400-406 (1990); Scott and Smith, Science 249:386-390 (1990); and U.S. Pat. No. 4,833,092 (Geysen), all incorporated herein by reference). A “small molecule” may be chemical compound, e.g., an organic compound, or a small peptide, or a larger peptide-containing organic compound or non-peptidic organic compound. A “small molecule” is not intended to encompass an antibody or antibody fragment. Although the molecular weight of small molecules is generally less than 2000 Daltons, this figure is not intended as an absolute upper limit on molecular weight.

Non-limiting examples of additional or alternative MS therapies for use in accordance with the present invention include, but are not limited to: fumaric acid salts, such as dimethyl fumarate; Sphingosine 1-phosphate (S1P)-antagonists, such as the S1B-blocking antibody Sphingomab; interferons, such as human interferon beta-1a (e.g., AVONEX® or Rebif®)) and interferon β-1b (BETASERON® human interferon β substituted at position 17; Berlex/Chiron); glatiramer acetate (also termed Copolymer 1, Cop-1; COPAXONE® Teva Pharmaceutical Industries, Inc.); an antibody or a fragment thereof (such as an antigen-binding fragment thereof), such as an anti-CD20 antibody, e.g., Rituxan® (rituximab), or an antibody or fragment thereof that competes with or binds an overlapping epitope with rituximab; mixtoxantrone (NOVANTRONE®, Lederle); a chemotherapeutic agent, such as clabribine (LEUSTATIN®), azathioprine (IMURAN®), cyclophosphamide (CYTOXAN®), cyclosporine-A, methotrexate, 4-aminopyridine, and tizanidine; a corticosteroid, such as methylprednisolone (MEDRONE®, Pfizer), or prednisone; CTLA4 Ig; alemtuzumab (MabCAMPATH®) or daclizumab (an antibody that binds CD25); statins; and TNF antagonists.

Glatiramer acetate is a protein formed from a random chain of amino acids (glutamic acid, lysine, alanine and tyrosine (hence GLATiramer)). Glatiramer acetate can be synthesized in solution from these amino acids at a ratio of approximately 5 parts alanine to 3 parts lysine, 1.5 parts glutamic acid and 1 part tyrosine using N-carboxyamino acid anhydrides.

Non-limiting examples of additional or alternative MS therapies for use in accordance with the present invention include, but are not limited to: antibodies or antagonists of other human cytokines or growth factors, for example, TNF, LT, IL-1, IL-2, IL-6, IL-7, IL-8, IL-12 IL-15, IL-16, IL-18, EMAP-11, GM-CSF, FGF, and PDGF. Still other exemplary agents include antibodies to cell surface molecules such as CD2, CD3, CD4, CD8, CD25, CD28, CD30, CD40, CD45, CD69, CD80, CD86, CD90 or their ligands. For example, daclizubmab is an anti-CD25 antibody that may ameliorate multiple sclerosis.

Still other exemplary antibodies include antibodies that provide an activity of an agent described herein, such as an antibody that engages an interferon receptor, e.g., an interferon beta receptor. Typically, in implementations in which the agent includes an antibody, it binds to a target protein other than VLA-4 or other than an α4 integrin, or at least an epitope on VLA-4 other than one recognized by natalizumab.

Still other exemplary agents include: FK506, rapamycin, mycophenolate mofetil, leflunomide, non-steroidal anti-inflammatory drugs (NSAIDs), for example, phosphodiesterase inhibitors, adenosine agonists, antithrombotic agents, complement inhibitors, adrenergic agents, agents that interfere with signaling by proinflammatory cytokines as described herein, IL-1β converting enzyme inhibitors (e.g., Vx740), anti-P7s, PSGL, TACE inhibitors, T-cell signaling inhibitors such as kinase inhibitors, metalloproteinase inhibitors, sulfasalazine, azathloprine, 6-mercaptopurines, angiotensin converting enzyme inhibitors, soluble cytokine receptors and derivatives thereof, as described herein, anti-inflammatory cytokines (e.g. IL-4, IL-10, IL-13 and TGF).

In some embodiments, an agent may be used to treat one or more symptoms or side effects of MS. Such agents include, e.g., amantadine, baclofen, papaverine, meclizine, hydroxyzine, sulfamethoxazole, ciprofloxacin, docusate, pemoline, dantrolene, desmopressin, dexamethasone, tolterodine, phenytoin, oxybutynin, bisacodyl, venlafaxine, amitriptyline, methenamine, clonazepam, isoniazid, vardenafil, nitrofurantoin, psyllium hydrophilic mucilloid, alprostadil, gabapentin, nortriptyline, paroxetine, propantheline bromide, modafinil, fluoxetine, phenazopyridine, methylprednisolone, carbamazepine, imipramine, diazepam, sildenafil, bupropion, and sertraline. Many agents that are small molecules have a molecular weight between 150 and 5000 Daltons.

Examples of TNF antagonists include chimeric, humanized, human or in vitro generated antibodies (or antigen-binding fragments thereof) to TNF (e.g., human TNF cc), such as D2E7, (human TNFα antibody, U.S. Pat. No. 6,258,562; BASF), CDP-571/CDP-870/BAY-10-3356 (humanized anti-TNFα antibody; Celltech/Pharmacia), cA2 (chimeric anti-TNFα antibody; REMICADE™, Centocor); anti-TNF antibody fragments (e.g., CPD870); soluble fragments of the TNF receptors, e.g., p55 or p75 human TNF receptors or derivatives thereof, e.g., 75 kd TNFR-IgG (75 kD TNF receptor-IgG fusion protein, ENBREL™; Immunex; see, e.g., Arthritis & Rheumatism 37:S295, 1994; J. Invest. Med. 44:235A, 1996), p55 kdTNFR-IgG (55 kD TNF receptor-IgG fusion protein (LENERCEPT™)); enzyme antagonists, e.g., TNFα converting enzyme (TACE) inhibitors (e.g., an alpha-sulfonyl hydroxamic acid derivative, WO 01/55112, and N-hydroxyformamide TACE inhibitor GW 3333, -005, or -022); and TNF-bp/s-TNFR (soluble TNF binding protein; see, e.g., Arthritis & Rheumatism 39:S284, 1996; Amer. J. Physiol.—Heart and Circulatory Physiology 268:37-42, 1995).

In one implementation, two or more agents are provided as a co-formulation. For example, in some embodiments, an anti-VLA-4 therapy and a second agent are provided as a co-formulation, and the co-formulation is administered to the subject. It is further possible, e.g., at least 24 hours before or after administering the co-formulation, to administer separately one dose of a first agent formulation and then one dose of a formulation containing a second agent. In another implementation, the first agent and the second agent are provided as separate formulations, and the step of administering includes sequentially administering the first agent and the second agent. The sequential administrations can be provided on the same day (e.g., within one hour of one another or at least 3, 6, or 12 hours apart) or on different days.

The first agent and the second agent each can be administered as a plurality of doses separately in time. The first agent and the second agent are typically each administered according to a regimen. The regimen for one or both may have a regular periodicity. The regimen for the first agent can have a different periodicity from the regimen for the second agent, e.g., one can be administered more frequently than the other. In one implementation, one of the first agent and the second agent is administered once weekly and the other once monthly. In another implementation, one of the first agent and the second agent is administered continuously, e.g., over a period of more than 30 minutes but less than 1, 2, 4, or 12 hours, and the other is administered as a bolus. The first agent and the second agent can be administered by any appropriate method, e.g., subcutaneously, intramuscularly, or intravenously.

In some embodiments, each of the first agent and the second agent is administered at the same dose as each is prescribed for monotherapy. In other embodiments, the first agent is administered at a dosage that is equal to or less than an amount required for efficacy if administered alone. Likewise, the second agent can be administered at a dosage that is equal to or less than an amount required for efficacy if administered alone.

### Cell Markers

Human hematopoietic cells can be characterized by various cell markers, as well as by gene expression analysis (Novershtern, et al. Cell 144(2):296-309, 2011, the contents of which are incorporated herein by reference). Cell markers for various hematopoietic cell populations include, but are not limited to, those provided below:

| | |
|---|---|
| CELL POPULATION | EXEMPLARY CELL MARKERS |

Hematopoietic Stem Cells

| | |
|---|---|
| HSC1 | lin−, CD133+, CD34dim |
| HSC2 | lin−, CD38−, CD34+ |

Ery Cells

| | |
|---|---|
| MEP | CD34+, CD38+, IL-3Rα−, CD45RA− |
| Ery1 | CD34+, CD71+, GlyA− |
| Ery2 | CD34−, CD71+, GlyA− |
| Ery3 | CD34−, CD71+, GlyA+ |
| Ery4 | CD34−, CD71^{lo}, GlyA+ |
| Ery5 | CD34−, CD71−, GlyA+ |

Mega Cells

| | |
|---|---|
| MEP | CD34+, CD38+, IL-3Rα−, CD45RA− |
| Mega1 | CD34+, CD41+, CD61+, CD45− |
| Mega2 | CD34−, CD41+, CD61+, CD45− |

Granulocyte/Monocyte Cells

| | |
|---|---|
| CMP | CD34+, CD38+, IL−3Rα^{lo}+, CD45RA− |
| GMP | CD34+, CD38+, IL−3Rα^{lo}+, CD45RA+ |
| Gran1 | CD34−, SSC^{hi}, CD45+, CD11b−, CD16− |
| Gran2 | CD34−, SSC^{hi}, CD45+, CD11b+, CD16− |
| Gran 3 | FSC^{hi}, SSC^{hi}, CD16+, CD11b+ |
| Mono1 | CD34−, CD33+, CD13+ |
| Mono2 | FSC^{hi}, SSC^{lo}, CD14+, CD45dim |
| Eos2 | FSC^{hi}, SSC^{lo}, IL3Rα+, CD33dim+ |
| Baso1 | FSC^{hi}, SSC^{lo}, CD22+, CD123+, |
| | CD33+/−, CD45dim |

DCs

| | |
|---|---|
| Denda2 | HLA DR+, CD3−, CD14−, CD16−, CD19−, |
| | CD56−, CD123−, CD11c+ |
| Denda1 | HLA DR+, CD3−, CD14−, CD16−, CD19−, |
| | CD56−, CD123+, CD11c− |

B Cells

| | |
|---|---|
| Pre-BCell2 | CD34+, CD10+, CD19+ |
| Pre-BCell3 | CD34−, CD10+, CD19+ |
| BCella1 | CD19+, IgD+, CD27− |
| BCella2 | CD19+, IgD+, CD27+ |
| BCella3 | CD19+, IgD−, CD27− |
| BCella4 | CD19+, IgD−, CD27+ |

NK Cells

| | |
|---|---|
| NKa1 | CD56−, CD16+, CD3− |
| NKa2 | CD56+, CD16+, CD3− |
| NKa3 | CD56−, CD16−, CD3− |
| NKa4 | CD14−, CD19−, CD3+, CD1d−F |

T Cells

| | |
|---|---|
| TCell2 | CD8+, CD62L+, CD45RA+ |
| TCell1 | CD8+, CD62L−, CD45RA+ |
| TCell3 | CD8+, CD62L−, CD45RA− |
| TCell4 | CD8+, CD62L+, CD45RA− |
| TCell6 | CD4+, CD62L+, CD45RA+ |
| TCell7 | CD4+, CD62L−, CD45RA− |
| TCell8 | CD4+, CD62L+, CD45RA− |

The global transcriptional profiles of each group of hematopoietic cells were determined, and are consistent with the established topology of hematopoietic differentiation (Novershtern, et al. Cell 144(2):296-309, 2011). In some embodiments, a gene associated with or specific for a cell type described herein, e.g., B cells, T cells, erythrocytes (e.g., early erythrocytes or late erythrocytes), granulocyte/monocyte progenitors, and hematopoietic stem cells, is a gene encoding the cell marker described herein. In some embodiments, a gene associated with or specific for a cell type described herein, e.g., B cells, T cells, erythrocytes (e.g., early erythrocytes or late erythrocytes), granulocyte/monocyte progenitors, and hematopoietic stem cells, can be determined, e.g., based on the co-expression the gene to be determined and one or more cell markers described herein.

### Gene Expression Assays

The methods described herein can include one or more steps of evaluating the expression levels of one or more genes, e.g., one or more genes described herein, e.g., one or more genes associated with, or specific for, a cell type, e.g., B cells, T cells, erythrocytes (e.g., early erythrocytes or late erythrocytes), granulocyte/monocyte progenitors, and hematopoietic stem cells. In some embodiments, the level of mRNA is determined. In some embodiments, the level of protein is determined. The level of mRNA or protein can be compared to a standard, e.g., a standard described herein.

The level of mRNA corresponding to a gene, e.g., a gene described herein, in a cell, e.g., a cell described herein, e.g. a B cell, a T cell, an erythrocyte (e.g., an early erythrocyte or a late erythrocyte), a granulocyte/monocyte progenitor, and a hematopoietic stem cell, can be determined, e.g., by in vitro or in situ formats.

Nucleic acid probes for the genes described herein can be used in hybridization or amplification assays that include, but are not limited to, Northern analyses, polymerase chain reaction analyses and probe arrays. One method for the detection of mRNA levels involves contacting the mRNA with a nucleic acid molecule (probe) that can hybridize to the mRNA encoded by the gene being detected. The nucleic acid probe can be, for example, a full-length nucleic acid for the gene being detected or a portion thereof, such as an oligonucleotide of at least 7, 10, 15, 30, 50, 100, 250 or 500 nucleotides in length and sufficient to hybridize under stringent conditions to the mRNA, cDNA, or portions thereof. The probes can be labeled with a detectable reagent to facilitate identification of the probe. Useful reagents include, but are not limited to, radioactivity, fluorescent dyes or enzymes capable of catalyzing a detectable product.

In one format, mRNA (or cDNA) is immobilized on a surface and contacted with the probes, for example by running the isolated mRNA on an agarose gel and transferring the mRNA from the gel to a membrane, such as nitrocellulose. In an alternative format, the probes are immobilized on a surface and the mRNA (or cDNA) is contacted with the probes, for example, in a two-dimensional gene chip array. A skilled artisan can adapt known mRNA detection methods for use in detecting the level of mRNA encoded by a gene described herein.

The level of mRNA in a sample that is encoded by a gene described herein can be evaluated with nucleic acid amplification, e.g., by RT-PCR (U.S. Pat. No. 4,683,202), ligase chain reaction (Barany, Proc. Natl. Acad. Sci. USA 88:189-193, 1991), self sustained sequence replication (Guatelli et al., Proc. Natl. Acad. Sci. USA 87:1874-1878, 1990), transcriptional amplification system (Kwoh et al., Proc. Natl. Acad. Sci. USA 86:1173-1177, 1989), Q-Beta Replicase (Lizardi et al., Bio/Technology 6:1197, 1988), rolling circle replication (U.S. Pat. No. 5,854,033), or any other nucleic acid amplification method, followed by the detection of the amplified molecules using techniques known in the art. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule including the nucleotide sequence flanked by the primers.

For in situ methods, a cell or tissue sample can be prepared/processed and immobilized on a support, typically a glass slide, and then contacted with a probe that can hybridize to mRNA that is encoded by the gene being analyzed.

A variety of methods can be used to determine the level of protein encoded by a gene, e.g., a gene described herein, in a cell, e.g., a cell described herein, e.g. a B cell, a T cell, an erythrocyte (e.g., an early erythrocyte or a late erythrocyte), a granulocyte/monocyte progenitor, and a hematopoietic stem cell. In general, these methods include contacting an agent that selectively binds to the protein, such as an antibody, with a sample to evaluate the level of protein in the sample. In one embodiment, the antibody includes a detectable label. Antibodies can be polyclonal or monoclonal. An intact antibody, or a fragment thereof (e.g., Fab or F(ab′)₂) can be used. The term “labeled,” with regard to the probe or antibody, is intended to encompass direct labeling of the probe or antibody by coupling (i.e., physically linking) a detectable substance to the probe or antibody, as well as indirect labeling of the probe or antibody by reactivity with a detectable substance. Examples of antibodies that can be used to detect a protein encoded by a gene described herein, e.g., a gene associated with or specific for B cells, T cells, erythrocytes (e.g., early erythrocytes or late erythrocytes), granulocyte/monocyte progenitors, and hematopoietic stem cells, are known in the art.

The detection methods for determining gene expression levels can also include methods which detect protein levels in a biological sample in vitro as well as in vivo. In vitro techniques for detection of protein include enzyme linked immunosorbent assays (ELISAs), immunoprecipitations, immunofluorescence, enzyme immunoassays (EIA), radioimmunoassays (RIA), and Western blot analysis. In vivo techniques for detection of proteins include introducing into a subject a labeled antibody against the protein. For example, the antibody can be labeled with a radioactive marker, e.g., a radioisotope) whose presence and location in a subject can be detected by standard imaging techniques. A radioisotope can be an α-, β-, or γ-emitter, or a β- and γ-emitter. Examples of radioisotopes that can be used include, but are not limited to: yttrium (⁹⁰Y), lutetium (¹⁷⁷Lu), actinium (²²⁵Ac), praseodymium, astatine (²¹¹At), rhenium (¹⁸⁶Re), bismuth (²¹²Bi or ²¹³Bi), and rhodium (¹⁸⁸Rh). Radioisotopes useful as labels, e.g., for use in diagnostics, include iodine (¹³¹I or ¹²⁵I), indium ¹¹¹In) technetium (⁹⁹mTc), phosphorus (³²P), carbon (¹⁴C), and tritium (³H).

### Correlative Functions

Some of the methods, systems and databases described herein feature correlative functions. The following section provides additional details, specific embodiments and alternatives for correlative functions. These are not limiting but are rather exemplary. They can optionally be incorporated into methods, databases, or systems described herein.

### Correlative Functions

A correlative function can relate X to Y, where X is a value for an element related to gene expression and Y is a value for an element related to the clinical score and allows adjustment of the value for X to select or identify a value for Y or the adjustment of the value for Y to select or identify a value for X. By way of example, X can be a value of gene expression level, a value of gene copy number, or a value of cell type, and in one or more of those cases, Y can be a clinical score associated with disease severity, disease progression, clinical outcome, or prognosis.

### Exemplary Computer Implementation

The methods and articles (e.g., systems or databases) described herein need not be implemented in a computer or electronic form. A database described herein, for example, can be implemented as printed matter.

Where appropriate, the systems and the functional operations described in this specification can be implemented in digital electronic circuitry, or in computer software, firmware, or hardware, including the structural means disclosed in this specification and structural equivalents thereof, or in combinations of them. The techniques can be implemented as one or more computer program products, i.e., one or more computer programs tangibly embodied in an information carrier, e.g., in a machine readable storage device or in a propagated signal, for execution by, or to control the operation of, data processing apparatus, e.g., a programmable processor, a computer, or multiple computers. A computer program (also known as a program, software, software application, or code) can be written in any form of programming language, including compiled or interpreted languages, and it can be deployed in any form, including as a standalone program or as a module, component, subroutine, or other unit suitable for use in a computing environment. A computer program does not necessarily correspond to a file. A program can be stored in a portion of a file that holds other programs or data, in a single file dedicated to the program in question, or in multiple coordinated files (e.g., files that store one or more modules, sub programs, or portions of code). A computer program can be deployed to be executed on one computer or on multiple computers at one site or distributed across multiple sites and interconnected by a communication network.

The processes and logic flows described in this specification can be performed by one or more programmable processors executing one or more computer programs to perform the described functions by operating on input data and generating output. The processes and logic flows can also be performed by, and apparatus can be implemented as special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, the processor will receive instructions and data from a read only memory or a random access memory or both. The essential elements of a computer are a processor for executing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. Information carriers suitable for embodying computer program instructions and data include all forms of non volatile memory, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

To provide for interaction with a user, aspects of the described techniques can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

The techniques can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation, or any combination of such back end, middleware, or front-end components. The components of the system can be interconnected by any form or medium of digital data communication, e.g., a communication network. Examples of communication networks include a local area network (“LAN”) and a wide area network (“WAN”), e.g., the Internet.

The computing system can include clients and servers. A client and server are generally remote from each other and typically interact through a communication network.

The relationship of client and server arises by virtue of computer programs running on the respective computers and having a client-server relationship to each other.

A number of implementations have been described. Nevertheless, it will be understood that various modifications can be made without departing from the spirit and scope of the described implementations. For example, the actions recited in the claims can be performed in a different order and still achieve desirable results. Accordingly, other implementations are within the scope of the following claims.

The invention is further illustrated by the following examples, which should not be construed as further limiting.

### EXAMPLES

### Example 1

### Patient Stratification in Secondary Progressive Multiple Sclerosis (SPMS) Using a Mixture of Experts Model

Multiple sclerosis (MS), particularly of the relapsing-remitting form (RRMS) has been extensively studied in the past years and various treatments have been developed. As of now, however, no therapy is effective against the more advanced stage of the disease, known as secondary progressive MS (SPMS). One of the major reasons for this is an increased heterogeneity in SPMS patients. In this example, the heterogeneity of SPMS represented by whole blood molecular profiles and clinical disease severity scores was examined.

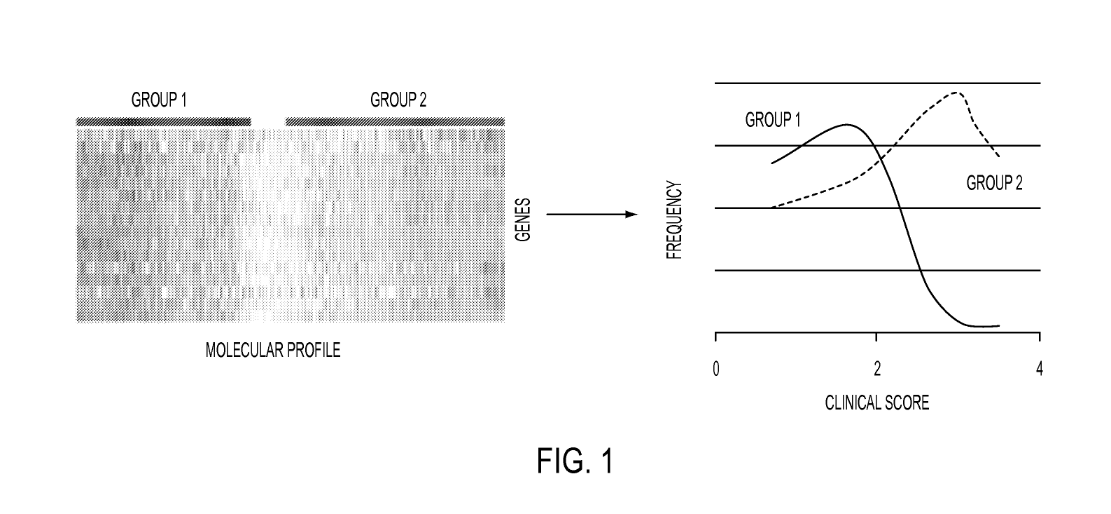

Traditionally, molecular profiles of patient cohorts are first analyzed independently of the disease score to identify molecularly uniform classes. Once distinct classes are identified, one checks for association with disease score or progression. This approach assumes that molecular subgroups directly reflect differences in disease severity and/or progression classes. An exemplary traditional paradigm of patient stratification is depicted in FIG. 1. In a recent paper Ottoboni et al. (Sci. Trans. Med., 4(153), 2012) applied this approach to blood profiles in a RRMS cohort and identified two molecular subclasses that correlate with time to relapse in RRMS patients treated with glatiramer acetate or IFNβ.

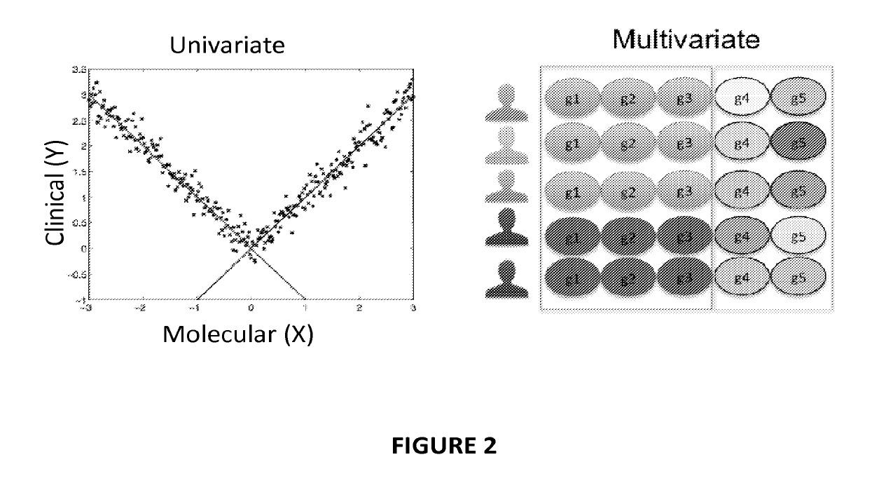

In this example, a new method for discovery of patient subclasses looking at the joint space of molecular markers and disease scores is described. The premise of this method is the supposition that in the joint space of molecular and disease scores, there may exist distinct subgroups of patients such that each group is characterized by a different dependence between molecular and disease scores. This concept is illustrated in a toy model in FIG. 2 (left) where a hypothetical relationship between the molecular variable X and clinical variable Y is depicted. Investigating just the space X of molecular markers or just the space Y of clinical variables does not reveal any subgroups. The underlying structure is only apparent in the joint X, Y space. When X and Y are multi-dimensional, as in the real world, de-convoluting the structure by looking at them individually becomes even harder.

In a multivariate setting (FIG. 2 (right)), there might be genes (g1, g2, g3) that are differentially expressed in the sub groups that act as the cleanest markers. However, different genes might be correlated with the disease score (or clinical outcome). The relationship between these genes and the clinical outcome may be different for the different subgroups, thereby indicating a difference in the underlying biology. This cannot be captured by traditional patient stratification methods that will tend to ignore g4 and g5 completely. Such complex relationships can be revealed by looking at the joint space of molecular and clinical variables.

The method described in this example looks at the joint distribution of patients in X (molecular) and Y (clinical) space, employs a mixture of linear models to explain the dependence between Y and X, and identifies optimal number of patient subgroups. Formally, given the number of clusters ‘K’, this is represented as the likelihood maximization problem:

$$Θ = {argmax}_{Θ} P ( Y , X )$$

$$P ( Y , X ) = {\sum }_{m = 1}^{K} P ( Y , X , {c}_{m} ) = {\sum }_{m = 1}^{K} P ( Y \vert X , {c}_{m} ) P ( X \vert {c}_{m} ) P ( {c}_{m} )$$

where Θ represents the set of parameters we use to describe the joint distribution. This includes parameters for the linear regression used to describe P(Y|X,cₘ), parameters for describing the clusters P(X|cₘ) and P(cₘ). To prevent overfitting, a regularized Expectation-Maximization (EM) algorithm was used to learn a sparse set of parameters and select the optimal number of clusters via the Bayesian Information Criterion (BIC).

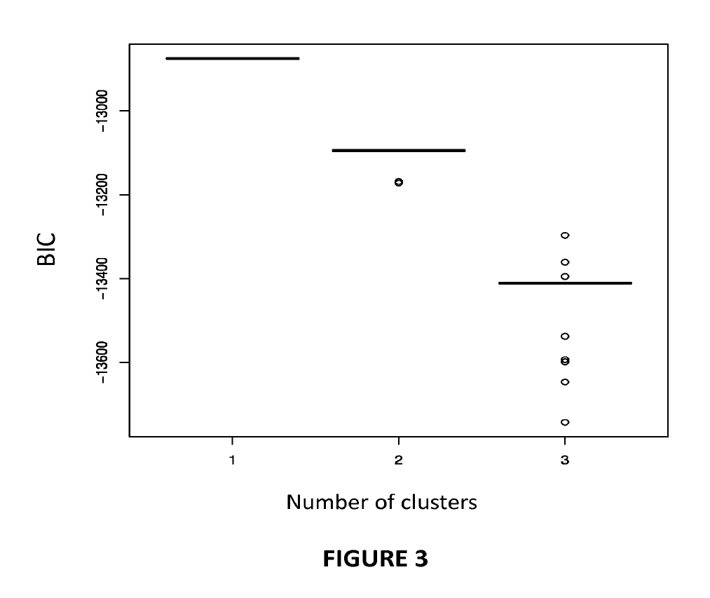

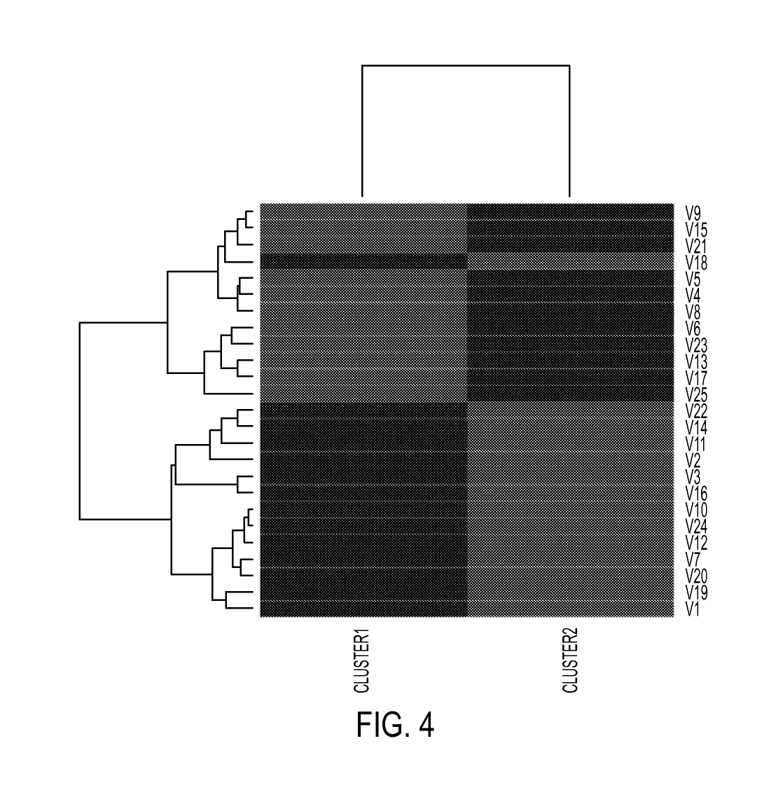

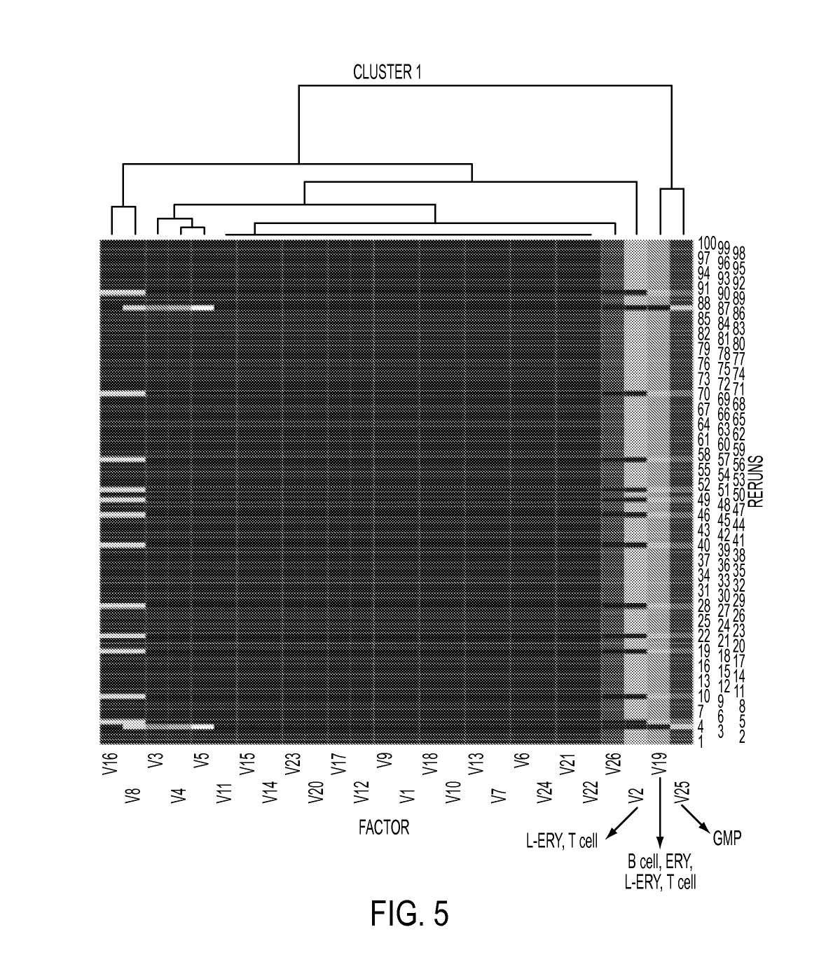

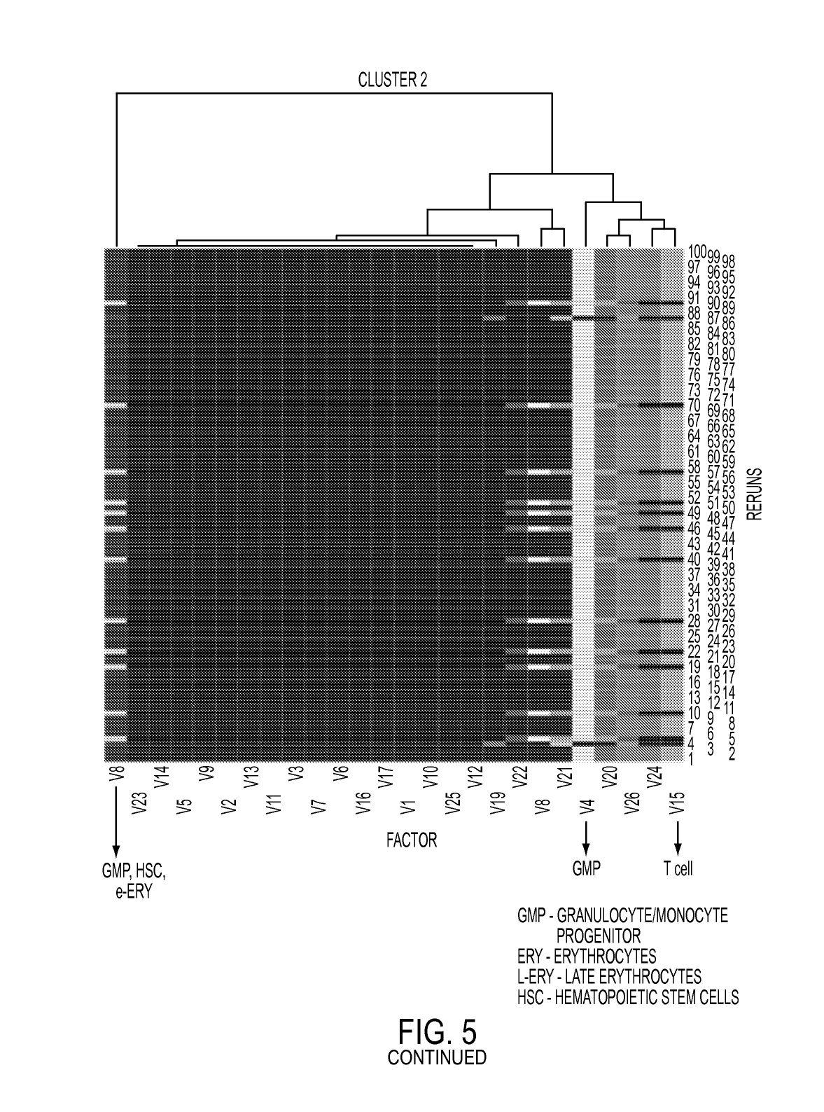

This approach was applied to the blood profiles of 190 SPMS patients characterized either by the Expanded Disability Status Scale (EDSS) or Multiple Sclerosis Severity Score (MSSS). Whole blood was collected in PAXGene tubes and profiled on Affymetrix platform hghgu133plusPM. Dimensionality of the molecular profiles was reduced by using non-negative matrix factorization (NMF). Number of reduced dimensions (factors) was chosen by maximizing cophenetic correlation. Twenty five factors are optimal in NMF. Two clusters are optimal—there were signs of overfitting at 3 clusters, as can be seen in FIG. 3. Clusters obtained were stable, as assessed through multiple runs of the algorithm.

Two subgroups of patients were distinctly identified by high or low expression of B cells, T cells, GMP cells and e-Erythrocyte (e-ERY) cells. Within each subgroup a different molecular signature best reflects the disease score. While in one subgroup B cells, GMP and ERY cells are correlated with disease severity (MSSS), in the other, GMP and ERY cells are correlated. It is important to note that relationships between the cell types and disease severity are different in the two subgroups. Also, since different features (or factors in the Non-negative matrix factorization) correlate with MSSS, different probes that are specific to the factors (but specific to similar cell types) correlate with MSSS. So, even though GMP is correlated in both sub groups, the actual probes that are correlated are different in both cases. Furthermore, lower BIC for our model indicates that the best model is better than the traditional approach that associates each signature with disease score in the entire cohort.

This example demonstrates, among other things, that multivariate biomarkers provide a new and useful approach for stratifying heterogeneous populations, e.g., MS patient populations. Different patient's sub-groups may be characterized by different dependence between molecular and clinical outcomes; thereby indicating different underlying disease biology. This can only be discovered by analyzing the joint space of molecular and clinical profiles. Traditional paradigm of patient stratification fails to capture this. Furthermore, in a multivariate setting, different features might be responsible for clustering and prediction of clinical outcome.

### REFERENCES

Gershenfeld, N., Schoner, B. and Metois, E., (1999) Cluster-weighted modelling for time-series analysis, Nature, 397, 329-332.
Jakkola, T., Machine Learning lecture notes, MIT.
Novershtern, N., Subramanian, A., Lawton, L., et al, (2011) Densely interconnected transcriptional circuits control cell states in human hematopoiesis, Cell, 144, 296-309.

### Example 2

### Patient Stratification in Multiple Sclerosis (MS)

### Summary

The objective of this Example is to identify subgroups of MS patients with more severe disease and distinct phenotypes. In particular, this Example aims to identify molecular characteristics of secondary progressive (SPMS) patients.

Towards this goal, the heterogeneity of MS represented by whole blood molecular profiles and clinical disease severity scores was examined. A new method for discovery of patient subclasses by looking at the joint space of molecular markers and disease scores was used. The premise of the method is that in this joint space, there may exist distinct subgroups of patients characterized by different dependencies between molecular and disease scores. The distribution of patients in the joint molecular (X) and clinical (Y) space was examined, a mixture of linear models was employed to explain the relationship between Y and X, and the optimal number of patient subgroups was identified. For molecular markers, genes differentially expressed in SPMS vs. healthy samples were considered.

This approach was applied to blood profiles of 190 SPMS patients characterized by Multiple Sclerosis Severity Score (MSSS). Two subgroups were identified within the cohort. Within each subgroup a different molecular signature best reflects the clinical score.

### Data Collection

The data used for this analysis are as follows: SPMS population (Accelerated Cure Project (ACP) group; n=106; molecular data: yes (whole blood); clinical data: yes; MRI: no; longitudinal: no; matched controls: yes (n=29); under treatment: yes).

### ProbeSelect

Due to the heterogeneity present in SPMS, naïve differential expression approaches do not yield any differentially expressed genes. A new algorithm, ProbeSelect, was developed to identify differentially expressed genes in heterogeneous populations. 1754 probes were selected based on the differential expression in SPMS-vs-control using this method.

The ProbeSelect algorithm computes a z-score for each probe for patient with respect to the means calculated from the healthy controls. For each probe, the algorithm counts the number of patients with absolute z-scores that are greater than the cutoff (e.g., 1.5). P value is used to quantify how likely these numbers of patients can be selected above the cutoff by chance. Probes that are statistically significant are selected after the P value is corrected for multiple hypothesis testing. ProbeSelect is described, e.g., in Hosur et al. Bioinformatics 30(4): 574-575, 2014.

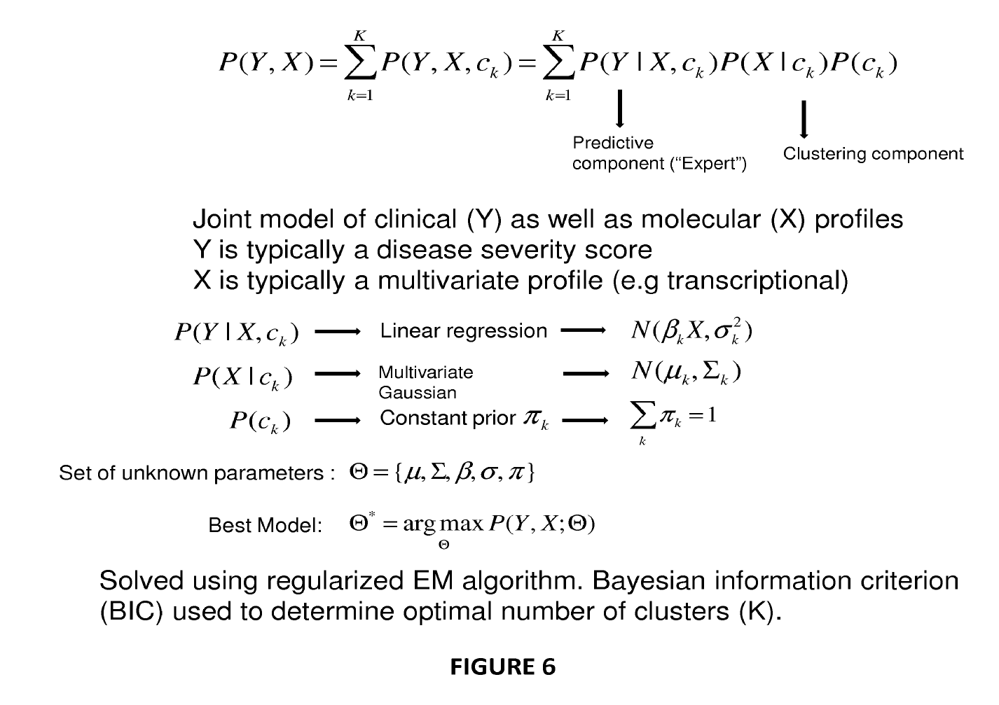

A mixture of experts approach was used to model joint distribution of molecular and clinical data. Differentially expressed genes from SPMS vs. healthy subjects were first reduced to a lower dimensional space using Non-negative Matrix Factorization (NMF). Each “factor” in the reduced dimensional space is a linear combination of the original differentially expressed genes. Sub-groups are characterized by different dependence between clinical and molecular variables. Different features can be important for stratification and clinical outcome. A brief illustration of the mixture of experts model for patient stratification is shown in FIG. 6.

### SPMS Subgroups

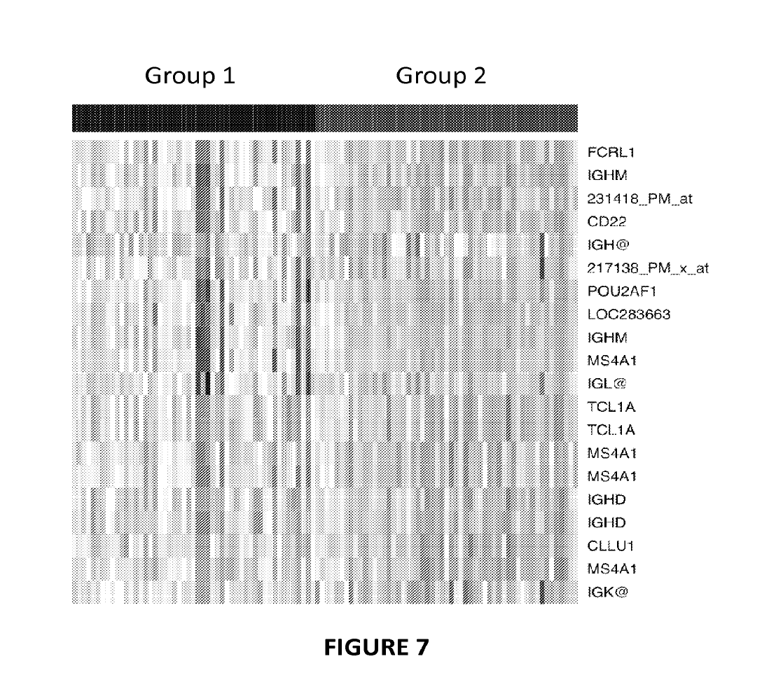

MSSS was used as the clinical variable. Two subgroups were found in the cohort: 51 and 55 patients in each subgroup. FIG. 7 shows the top 20 differentially expressed probes between ACP (SPMS) subgroups. The differentially expressed genes or loci include, e.g., FCRL1, IGHM, 231418_PM_at, CD22, IGH@, 217138_PM_x_at, POU2AF1, LOC283663, IGHM, MS4A1, IGL@, TCL1A, MS4A1, IGHD, CLLU1, and IGK@.

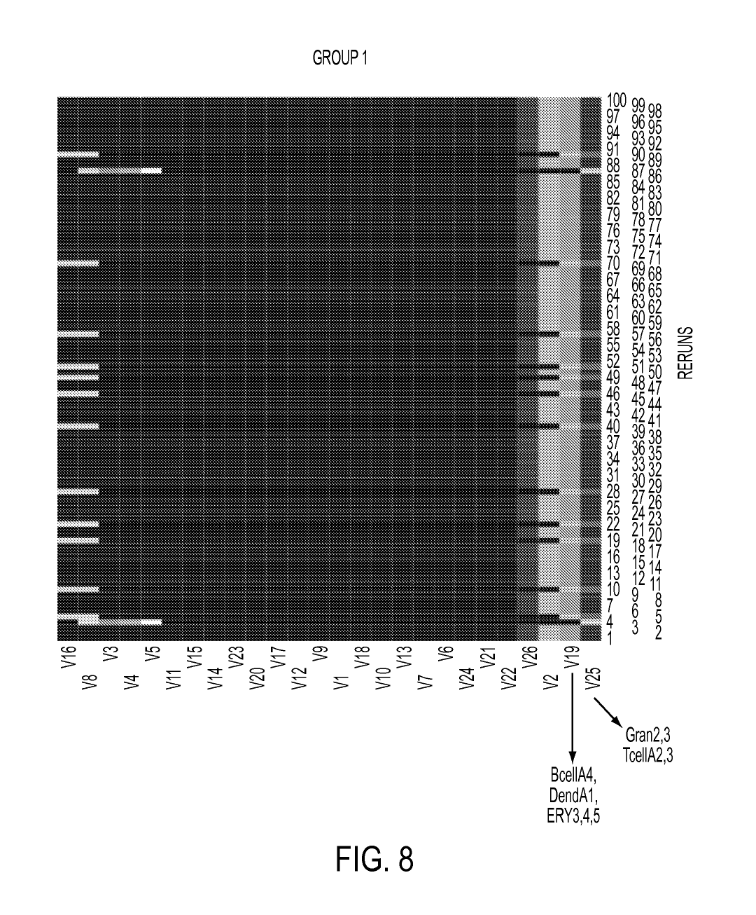

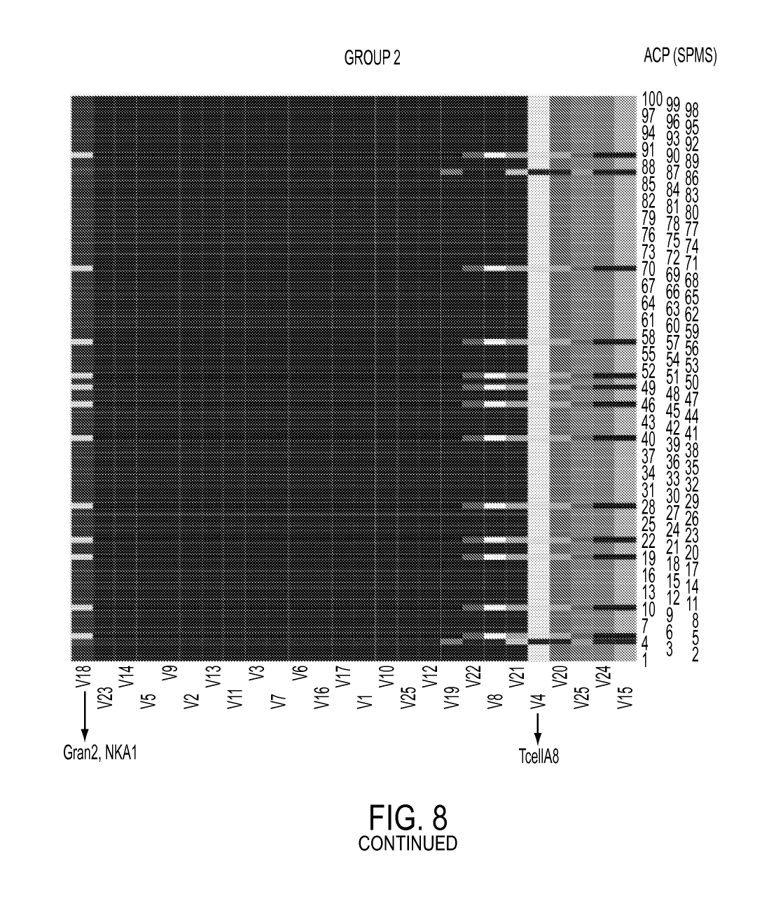

As shown in FIG. 8, there is different dependence between molecular factors and disease severity in the subgroups. The factors (or probes) were mapped to different cell types as described above. For example, B cells (type A4), dendritic cells (type A1), erythrocytes (types 3, 4 and 5), and T cells (types A2 and A3) were identified in cluster 1; and granulocytes, NK cells (type A1), and T cells (type A8) were indicated in cluster 2.

### Conclusions

Multivariate biomarkers provide a new approach to stratify heterogeneous populations. Different subgroups were characterized by different dependence between molecular and clinical outcomes; thereby indicating different underlying biology. This can only be discovered by analyzing the joint space of molecular and clinical profiles. Traditional paradigm of patient stratification fails to capture this. In a multivariate setting, different features might be responsible for clustering and predictive of clinical outcome.

### Example 3

### ExpertMIX Stratification: A Method for Integrated Modeling of Clinical and Molecular Disease Variability

### Introduction

The objective of this Example is to model clinically observed variability along with the molecular variability of patient samples and provide an integrated perspective on molecular aspects of disease. A better understanding of the underlying causes and factors contributing to disease variability will provide patients with better prognosis and more effective treatment algorithms. Different clinical measures are employed to describe state or prognosis depending on treatment or monitoring objective. Clinical variability can be defined, for example, as varied disease severity, faster or slower disease progression, or different and unpredictable response to therapy.

This Example illustrates a new method (sometimes referred to herein as “expertMIX”) for discovery of patient subclasses looking at the joint space of molecular markers and disease scores. The algorithm described below was formulated for the case when the distribution of the clinical scores is assumed to be approximately normal. The algorithm also assumes that the molecular characteristics describing uniform group are normally distributed and there is linear relationship between clinical score and some of the molecular characteristics.

The expertMIX method was applied to the analysis of Secondary Progressive MS (SPMS) patient profiles. SPMS is a more advance stage of the MS, i.e., for majority of patients the disease starts as a relapsing remitting form of the disease. This method identified two distinct SPMS patients' subgroups, A and B, and provided molecular insights into clinical variability of SPMS. The two SPMS subgroups are significantly different in the expression levels of B cell signature.

The expertMIX method also revealed additional signatures associated with disease severity variability in SPMS. These signatures reflect the association of other immune cell-types, such as NK cells, dendritic cells, granulocytes and T cells, with disease severity in different patient subgroups.

### Mixture of Expert Method

ExpertMIX method is at the core of an approach to identify and interpret clinical and molecular variability. This approach follows four steps outlined as follows: (A) feature selection using ProbeSelect (Hosur et al. Bioinformatics 30(4): 574-575, 2014); (B) identification of non-redundant feature representation, i.e., dimensionality reduction using NMF (Lee and Seung Nature 401(6755):788-791, 1999); (C) identification of molecular subgroups and features associated with clinical variability using expertMIX; and (D) molecular characterization of the subgroups and biological interpretation of features associated with clinical variability.

The algorithm explores the hypothesis that k=1, . . . , m subgroups of samples have different molecular characteristics associated with the clinical variability. For each k the Bayesian Information Criterion (BIC) assesses the optimal mixture of expert models explaining disease variability in entire set. Details of the optimization procedure for k-mixtures of experts are provided in Materials and Methods. Median BIC(k) is compared to the BIC(k−1) and the algorithm finds m=k such that the median(BIC(m))>median(BIC(m−1)). The distribution plots for k=1, . . . , m show stability of different mixture of experts models. Thus, in the final model selection value of the median BIC(k) and its variability can be considered.

### Results

To investigate clinical variability of Secondary Progressive MS (SPMS) and molecular signatures that may associate with such variability, a set of 116 SPMS patient whole blood samples from the Accelerated Cure Projects were analyzed. In the study, MSSS (Roxburgh et al. Neurology 64(7):1144-1151, 2005) represented the clinical variability score. MSSS was chosen for this investigation since EDSS (Rudick et al. Arch Neurol 67(11): 1329-1335, 2010), a measure of disease severity commonly used in MS assessment, is not normally distributed and thus less suited for the linear representation of molecular-clinical association. Additionally, the MSSS captures the longitudinal aspects of disease severity, while EDSS represents the current status.

Gene expression was measured using the Affymetrix htHGU133plusPM array and protocols are described in the Materials and Methods. First, 116 SPMS patients' profiles were compared to 30 profiles of gender and age matched healthy controls. 1753 transcripts that are significantly expressed in disease vs. controls were selected using the ProbeSelect method (Hosur et al. Bioinformatics 30(4): 574-575, 2014). Second, since gene expressions are correlated, the Nonnegative Matrix Factorization (NMF) (Lee and Seung Nature 401(6755):788-791, 1999) was applied to identify independent components among the 1753 transcripts. Using the cophenetic measure as a metric for evaluating the dimensionality reduction (Ottoboni et al. Sci Transl Med 4(153):153ra131, 2012; Hofree et al. Nat Methods 10(11): 1108-1115, 2013), 25-factors representing independent molecular signals across the 116 disease profiles were selected. The selected molecular factors together with MSSS score are the input to the expertMIX algorithm.

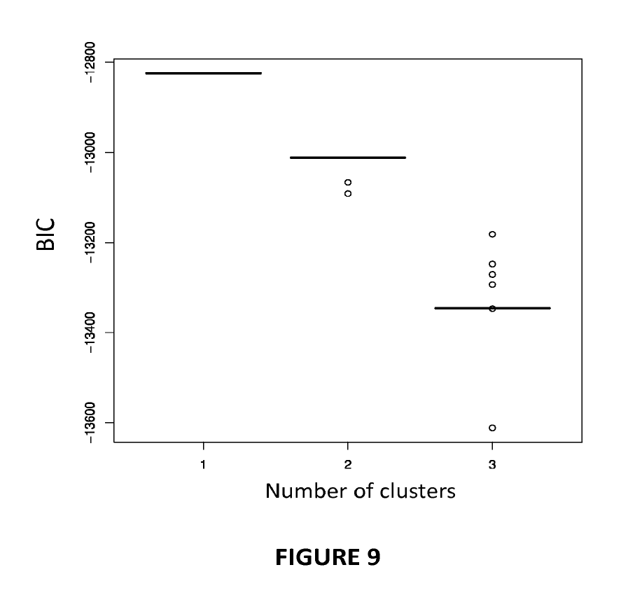

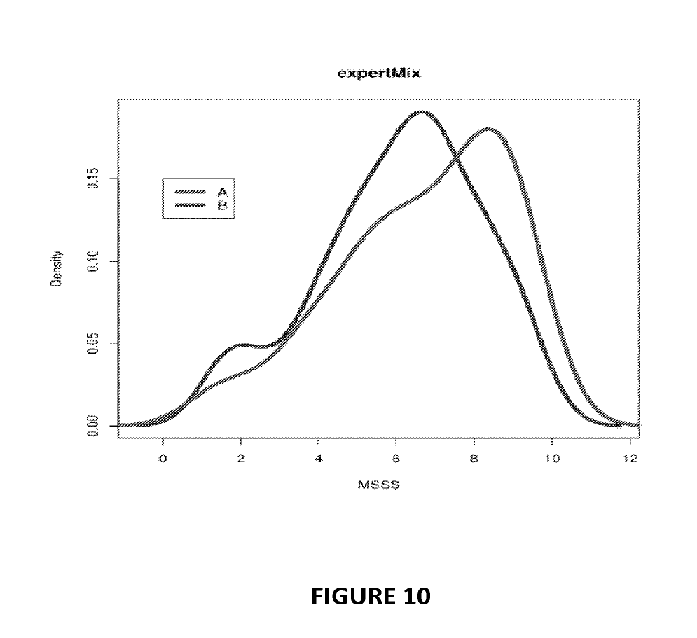

First, the expertMIX approach was applied to the SPMS cohort and discovered two subgroups of samples differentiated by their molecular/clinical characteristics. As shown in FIG. 9, the BIC distributions for 1 to 3 mixture-of-experts models were used to explain molecular-clinical relationship. The BIC measure indicates that three molecular subgroups best explain the MSSS variability in this data set. However, due to a relatively small number of samples the 3-experts model is more variable than 2-experts model. Thus, a lower complexity and more stable 2-experts model was chosen to represent clinical and molecular variability in SPMS. Lower BIC for 2-experts model in comparison to just one indicates that the best model is better than the traditional approach associating molecular factors with MSSS score across the entire cohort. The top 20 transcripts separating cluster A from B are shown in FIG. 7. As shown in FIG. 10, the distribution of MSSS is shifted towards higher MSSS in the SPMS_{A}. Kruskal Wallis p value=0.122. With the traditional approach of unsupervised clustering one can also assign two groups. However, there is no significant difference in the MSSS distributions for these clusters.

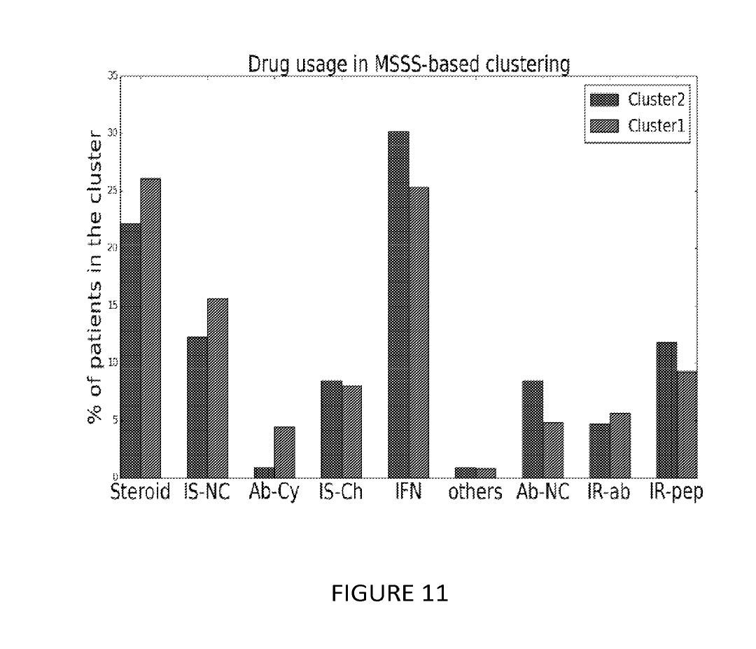

Given that SPMS patients are treated with different therapies, it was investigated whether in the SPMS cohort the SPMS_{A} and SPMS_{B} subgroups are associated with different treatments. No such association was found (see FIG. 11).

In addition to identifying the molecular subgroups, the method described in this example also identified the molecular factors that may explain clinical variability in these subgroups. Factors significantly associated with clinical variability were determined as described in the Methods. To provide a biological interpretation of molecular factors associating with MSSS score, a GeneSet enrichment-like approach was employed. The D-MAP (differentiation map of hematopoiesis) (Novershtern et al. Cell 144(2):296-309, 2011) data was used to find which immune cell types contribute to factors associated with the clinical variability. For example, the expertMIX found that in the SPMS_{A} subgroup the MSSS score variability is correlated with expression of transcripts representing, for example, B cell (type A4), dendrocyte (type A1), erythrocyte (types 3, 4 and 5), granulocyte (types 2 and 3), T cell (types A2 and A3) lineages. There appears no such clear linear relationship between MSSS and molecular factors for group SPMS_{B}.

Currently, no therapy is effective against the advanced stage of MS, the secondary progressive MS (SPMS). One of the reasons for this is an increased heterogeneity in SPMS patients. The findings of B-Cell signature as the molecular marker differentiating SPMS patients' subgroups suggest testing efficacy of B-cell depleting therapies (e.g., RITUXAN) for SPMS treatment. However, identification of a subgroup of SPMS patients with higher expression of B-cell signature indicates that B-cell depletion may not be the right therapy for every patient and molecular markers may help identify suitable target population.

### Materials and Methods

### Patients and Samples

116 whole blood samples from patients diagnosed with SPMS were obtained from the Accelerated Cure Project (ACP) that is an observational cohort. Patients are under different treatments in prior year as well as at the time of sample collection. Additional samples from 30, age and gender matched healthy controls, was also provided by the ACP.

### RNA Isolation, Labeling, Hybridization and Scanning

RNA was isolated from PaxGene tubes as per the manufacturer's standard protocol. RNA was quantitated using the Nanodrop (Nanodrop Technologies, Willmington, Del.) and the quality was assessed using the Agilent 2100 Bioanalyzer (Agilent Technologies, Palo Alto, Calif.). 25 ng of Paxgene purified total RNA was amplified, fragmented and labeled using FL Ovation cDNA biotin automated module V2 (cat #A4200, Nugen inc., San Carlos Calif.) and FL Ovation cDNA biotin automated module V2 (cat #A4200, Nugen inc., San Carlos Calif.) using Beckman Arrayplex automated workstation according to manufactures protocol. 3 ug of fragmented and labeled cDNA was hybridized HTHGU-133plusPM arrays.

Washing, staining and scanning of the hybridized arrays was completed as described in the Eukaryotic Target Preparation protocol in the Affymetrix expression analysis technical manual (702064 rev 2) for Genechip® cartridge arrays using the Genechip® Array Station (Affymetrix, Santa Clara, Calif.).

### Data Normalization

Data was normalized using the GCRMA within SPMS and control samples.

Optimization of the k-Mixture of Experts Model

The method described in this example looks at the joint distribution of patients in X (molecular) and Y (clinical) space. It uses a mixture of k-linear models to explain the dependence between Y and X, and identifies optimal number of patient subgroups. Formally, given the number of subgroups ‘k’, we find the optimal models by maximization of the likelihood:

$$Θ = {argmax}_{Θ} P ( Y , X ) \\ P ( Y , X ) = {\sum }_{m = 1}^{K} P ( Y , X , {c}_{m} ) = {\sum }_{m = 1}^{K} P ( Y \vert X , {c}_{m} ) P ( X \vert {c}_{m} ) P ( {c}_{m} ) \qquad ( 1 )$$

where Θ represents the set of parameters describing joint distribution. A linear dependence between the clinical variable Y and molecular variables X is assumed. The choice of the expert model P(Y|X,cₘ) depends on the distribution of Y. In principle, any generalized linear model can be used to model the clinical variable Y. Similarly the choice of the P(X|cₘ) depends on the molecular variables distribution. X is assumed to be normally distributed (multivariate) within each subgroup. P(c)-π it is the prior distribution for the subgroups. Fitness of the model made of k experts is evaluated using BIC (Bayesian information Criterion).

Θ includes parameters for the linear regression used to describe P(Y|X,cₘ)=N(βX,σ²) where σ is the random error, parameters for describing the distribution in m-subgroups P(X|cₘ) and P(cₘ). P(X|cₘ)=N(μ, Σ) is a multivariate Gaussian distribution.

To prevent over-fitting, a regularized Expectation-Maximization algorithm (EM) is used to solve the maximization problem for linear experts in multi-variable molecular space.

The following describes the EM algorithm for the joint model for log likelihood.

In the expectation step (E-step) the posterior probability is calculated as follows:

$${∀}_{k} P ( {c}_{k} \vert x , y ) ∝ P ( y \vert x , {c}_{k} ) P ( x \vert {c}_{k} ) P ( {c}_{k} )$$

For the M-step, we maximize the expected value of the complete log-likelihood:

$$\begin{matrix}Q = {E}_{c \vert X , Y} log P ( X , Y , c ) \\ = {E}_{c \vert X , Y} log \{ P ( Y \vert X , c ) P ( X \vert c ) P ( c ) \} = \\ = {E}_{c \vert X , Y} log P ( Y \vert X , c ) + {E}_{c \vert X , Y} log P ( X \vert c ) + {E}_{c \vert X , Y} log P ( c ) 22\end{matrix} \qquad ( 2 )$$

The maximization step is performed through first order derivative of Q with respect to parameters Θ={β, μ, Σ, π}.

$$\frac{\partial Q}{\partial \{ β , μ , σ , π \}} = 0 \qquad ( 3 )$$

Each component in the equation (2) above depends only on one set of parameters Θ and equation (3) simplifies to:

$$\frac{\partial {E}_{c \vert X , Y} log P ( Y \vert X , c )}{\partial β} = 0 \\ \frac{\partial {E}_{c \vert X , Y} log P ( X \vert c )}{\partial ( μ , σ \}} = 0 \\ \frac{\partial {E}_{c \vert X , Y} log P ( X \vert c )}{\partial π} = 0 \qquad ( 4 )$$

Thus maximization step of the EM algorithm is solved by 3 independent maximizations:

1) In the linear expert maximization term we assume the following prior for the parameters to induce sparsity (similar to LASSO approach, Tipping and Faul 2000)

$$P ( β ) = {\prod }_{d + 1} N ( 0 , {α}_{i}^{- 1} )$$

To learn parameters of the experts, the Relevance Vector formalism is employed as detailed in Tipping and Faul 2001. Each expert is fit to the entire dataset, with each data point weighted by the posterior probability of the data point belonging to the cluster.

2) For the maximization of the Gaussian mixture term, we also assume a prior distribution for the covariance matrix. If N(μ, Σ) is the multivariate Gaussian distribution for one cluster, for the covariance matrix we assume the inverse Wishart distribution as a prior:

$$P ( Σ ) = {\vert Σ \vert}^{- m / 2} exp ( - \frac{m}{2} tr ( S {Σ}^{- 1} ) )$$

where, the matrix ‘S’ is the covariance matrix of the initial clusters obtained through k-means. ‘m’ is set to 1 in the computations.

Evaluation of the mixture model is done with the Bayesian Information Criterion.

### Evaluation of the Standard Approach Model

In order to compare expertMix approach with the traditional clustering and the evaluation of the clinical differences between clusters we calculated the BIC as follows. First the clusters defined in molecular space are found using k-means and clusters means and covariance matrix is calculated. Means and standard deviation for the disease severity scores are calculated for each cluster cₖ. The data model then is described by Gaussian distributions of X and Y separately for given number of clusters.

$$P ( Y , X ) = {\sum }_{k = 1}^{K} P ( Y , X , {c}_{k} ) = {\sum }_{k = 1}^{K} P ( Y \vert {c}_{k} ) P ( X \vert {c}_{k} ) P ( {c}_{k} )$$

Average BIC for 100 initializations of k-means clustering is calculated.

### Transcripts Differentiate the Subgroups of Patients

In order to identify transcripts defining molecular subgroups of patients, standard differential expression analysis was performed as implemented by the LIMMA package (Smyth, Stat Appl Genet Mol Biol 3: Article3, 2004). Probes with p<0.05 after FDR correction and those that are at least 1.3-fold different between the groups as differentially expressed were selected.

Factors that Associate with Disease Severity

For each number of sample subgroup k, and each iteration we record the linear coefficient βⱼ representing relationship between clinical variable Y and molecular factor Xⱼ. Where j=1 . . . J is the number of molecular features considered as possibly associated with clinical variability. In each optimization repeat, LASSO-like linear regression selects out the non-significant factors by setting respective coefficient to zero, while significant factors have non-zero βⱼ. We calculate mean of the linear coefficients for each factor mean (βⱼ) over the 100 iterations. The factors that have coefficient significantly different from zero (one-tailed t-test p.value<0.05) are called significantly associated with disease severity.

### Biological Interpretation of the Factors

Each factor is interpreted in terms of enriched cell-types using the DMAP data (Novershtern et al. Cell 144(2):296-309, 2011). DMAP data is used to define signatures for each cell-type. First, we select transcripts that are differentially expressed specifically in that cell type. Each probe's contribution to a factor is then converted to a probability by normalizing over the factors. Probes that are specific to a factor have a higher probability. Next, a gene-set enrichment analysis (instead of correlation in the GSEA enrichment score, a constant of 1 is used) is carried out for each factor to determine enrichment of the cell-type signatures. To assess the significance of the enrichment score, permutation p-values were calculated for each cell type, and significant cell-types are selected as the interpretation of the factor.

### EQUIVALENTS

Those skilled in the art will recognize, or be able to ascertain using no more than routine experimentation, many equivalents to the specific embodiments of the invention described herein. Such equivalents are intended to be encompassed.

1. A method of treating a subject having multiple sclerosis (MS) or at risk of developing secondary progressive multiple sclerosis (SPMS), or treating or preventing one or more symptoms associated with MS in a subject having MS, or at risk of developing SPMS, the method comprising:
administering an MS therapy to a subject having MS or at risk of developing SPMS,
wherein the subject has two or more genes associated with B cells down-regulated by at least about 10% compared to expression levels of the same genes in B cells in a normal subject, two or more genes associated with T cells down-regulated by at least about 10% compared to expression levels of the same genes in T cells in a normal subject, two or more genes associated with early erythrocytes (e-ERYs) down-regulated by at least about 10% compared to expression levels of the same genes in e-ERYs in a normal subject, and two or more genes associated with granulocyte/monocyte progenitors (GMPs) up-regulated by at least about 0.5 fold compared to expression levels of the same genes in GMPs in a normal subject; or
wherein the subject has two or more genes associated with B cells up-regulated by at least about 0.5 fold compared to expression levels of the same genes in B cells in a normal subject, two or more genes associated with T cells up-regulated by at least about 0.5 fold compared to expression levels of the same genes in T cells in a normal subject, two or more genes associated with early erythrocytes (e-ERYs) up-regulated by at least about 0.5 fold compared to expression levels of the same genes in B cells in a normal subject, and two or more genes associated with granulocyte/monocyte progenitors (GMPs) down-regulated by at least about 10% compared to expression levels of the same genes in GMPs in a normal subject.

2. The method of claim 1, comprising acquiring knowledge that a subject has two or more genes associated with B cells down-regulated, two or more genes associated with T cells down-regulated, two or more genes associated with early erythrocytes (e-ERYs) down-regulated, and two or more genes associated with granulocyte/monocyte progenitors (GMPs) up-regulated; or
acquiring knowledge that a subject has two or more genes associated with B cells up-regulated, two or more genes associated with T cells up-regulated, two or more genes associated with early erythrocytes (e-ERYs) up-regulated, and two or more genes associated with granulocyte/monocyte progenitors (GMPs) down-regulated, and, based upon that knowledge, administering the subject an MS therapy, and,
based upon that knowledge, administering the subject an MS therapy.

3.-6. (canceled)

7. The method of claim 1, wherein the gene associated with B cells is a B cell-specific gene, the gene associated with T cells is a T cell-specific gene, the gene associated with e-ERYs is an e-ERY-specific gene, or the gene associated with GMPs is a GMP-specific gene.

8.-10. (canceled)

11. The method of claim 1, wherein the MS therapy comprises one or more of: an anti-VLA-4 therapy, an anti-IL-2 receptor therapy, an interferon beta, a sphingosine 1-phosphate (S1P) antagonist, or glatiramer acetate (GA).

12. The method of claim 11, wherein:
the anti-IL-2 receptor therapy comprises, e.g., daclizumab;
the interferon beta comprises interferon beta-1a or interferon beta-1b; or
the sphingosine 1-phosphate (S1P) antagonist comprises fingolimod.

13.-15. (canceled)

16. The method of claim 1, wherein the subject has been treated with an MS therapy.

17. The method of claim 1, further comprising acquiring a sample from the subject.

18. The method of claim 17, further comprising determining the expression levels of two or more genes associated with B cells, two or more genes associated with T cells, two or more genes associated with e-ERYs, and two or more genes associated with GMPs, in the sample, and comparing the expression levels of the genes with expression levels of the same genes in the same cell types in a normal subject, wherein the expression levels are determined prior to initiating, during, or after, a treatment in the subject, or is at the time of diagnosis of the subject with MS.

19.-20. (canceled)

21. The method of claim 18, wherein the expression levels of the genes are determined by oligonucleotide array or quantitative RT-PCR.

22. (canceled)

23. The method of claim 1, further comprising selecting an MS therapy, or selecting or identifying a subject having two or more genes associated with B cells down-regulated, two or more genes associated with T cells down-regulated, two or more genes associated with e-ERYs down-regulated, and two or more genes associated with GMPs up-regulated, for treatment with an MS therapy; or
selecting an MS therapy, or selecting or identifying a subject having two or more genes associated with B cells up-regulated, two or more genes associated with T cells up-regulated, two or more genes associated with early erythrocytes (e-ERYs) up-regulated, and two or more genes associated with granulocyte/monocyte progenitors (GMPs) down-regulated, and, based upon that knowledge, for treatment with an MS therapy.

24. The method of claim 23, wherein the subject is already receiving an MS therapy and the identification of the down-regulation or up-regulation of the genes indicates that the subject can receive an alternative MS therapy, or that the subject should stop receiving the MS therapy, or the dose or dosing schedule of the MS therapy should be altered.

25. (canceled)

26. The method of claim 1, further comprising:
identifying a clinical outcome of the subject having two or more genes associated with B cells, two of more genes associated with GMPs, and two or more genes associate with ERYs, up-regulated or down-regulated, wherein the up-regulation or down-regulation is correlated with or indicative of a clinical score associated with disease severity, disease progression, clinical outcome, or prognosis, or
determining a clinical score associated with disease severity, disease progression, clinical outcome, or prognosis, for the subject, wherein the clinical score comprises Expanded Disability Status Scale (EDSS), or Multiple Sclerosis Severity Score (MSSS).

27.-58. (canceled)

59. A method of identifying a subject having multiple sclerosis (MS) or at risk of developing secondary progressive multiple sclerosis (SPMS), for treatment with an MS therapy, or evaluating or monitoring disease progression in a subject having MS or at risk of developing SPMS, the method comprising:
providing a sample from a subject having MS or at risk of developing SPMS,
determining the expression levels of two or more genes associated with B cells, two or more genes associated with T cells, two or more genes associated with e-ERYs, and two or more genes associated with GMPs, in the sample;
comparing the expression levels of the genes with expression levels of the same genes in the same cell type in a normal subject; and
identifying the subject for treatment with an MS therapy, or evaluating or monitoring disease progression,
on the basis that the subject has two or more genes associated with B cells down-regulated, two or more genes associated with T cells down-regulated, two or more genes associated with e-ERYs down-regulated, and two or more genes associated with GMPs up-regulated; or
on the basis that the subject has two or more genes associated with B cells up-regulated, two or more genes associated with T cells up-regulated, two or more genes associated with e-ERYs up-regulated, and two or more genes associated with GMPs down-regulated.

60.-64. (canceled)

65. The method of claim 1, further comprising generating a personalized MS treatment report, by obtaining a sample from a subject having MS, determining the expression levels of two or more genes associated with B cells, two or more genes associated with T cells, two or more genes associated with e-ERYs, and two or more genes associated with GMPs, and selecting an MS therapy, based on the expression levels identified, down-regulation of two or more genes associated with B cells, down-regulation of two or more genes associated with T cells, down-regulation of two or more genes associated with early erythrocytes (e-ERYs), and up-regulation of two or more genes associated with granulocyte/monocyte progenitors (GMPs) indicates a first course of treatment; and up-regulation of two or more genes associated with B cells, up-regulation of two or more genes associated with T cells, up-regulation of two or more genes associated with early erythrocytes (e-ERYs), and down-regulation of two or more genes associated with granulocyte/monocyte progenitors (GMPs) indicates a second different course of action.

66. The method of claim 1, further comprising determining a gene expression profile for a subject having MS, comprising:
directly acquiring knowledge of the expression levels of two or more genes associated with B cells, two or more genes associated with T cells, two or more genes associated with e-ERYs, and two or more genes associated with GMPs in a sample from a subject having MS, and
responsive to a determination of down-regulation or up-regulation of the genes, one or more of:
(1) stratifying a subject population;
(2) identifying or selecting the subject as likely or unlikely to respond to an MS therapy;
(3) selecting an MS therapy;
(4) treating the subject; or
(5) prognosticating the time course and/or severity of the disease in the subject.

67. The method of claim 66, wherein responsive to the direct acquisition of knowledge of the expression levels of the genes, the subject is classified as a candidate to receive an MS therapy or is identified as likely to respond to an MS therapy.

68. (canceled)

69. The method of claim 1, further comprising producing a reaction mixture comprising:
a plurality of detection reagents, or purified or isolated preparation thereof; and
a target nucleic acid preparation derived from a sample from a subject having multiple sclerosis (MS),
wherein said plurality of detection reagents can determine expression levels of: two or more genes associated with B cells, two or more genes associated with T cells, two or more genes associated with e-ERYs, and two or more genes associated with GMPs.

70. The method of claim 59, further comprising evaluating a subject population having MS by a system, wherein the system comprises at least one processor operatively connected to a memory, the at least one processor has:
a first plurality of values for a plurality of subjects having MS, wherein each value is indicative of expression of a gene associated with T cells, B cells, e-ERYs, or GMPs;
a second plurality of values for the plurality of subjects having MS, wherein each value is indicative of a clinical score associated with disease severity, disease progression, clinical outcome, or prognosis for a subject having MS; and
a function that correlates the first plurality of values with the second plurality of values to provide an output of classification of the MS of the subject population.

71. The method of claim 70, wherein the correlative function determines the joint distribution of the plurality of the subjects in a space of gene expression (X) and clinical score (Y) by the likelihood maximization problem:
$$Θ = {argmax}_{Θ} P ( Y , X )$$
$$P ( Y , X ) = {\sum }_{m = 1}^{K} P ( Y , X , {c}_{m} ) = {\sum }_{m = 1}^{K} P ( Y \vert X , {c}_{m} ) P ( X \vert {c}_{m} ) P ( {c}_{m} )$$
where Θ represents the set of parameters used to describe the joint distribution, which includes parameters for the linear regression used to describe P(Y|X,cₘ), parameters for describing the clusters P(X|cₘ) and P(cₘ), or the correlative function uses a regularized Expectation-Maximization algorithm (EM) to learn a sparse set of parameters; or
wherein the output indicates an optimal number of clusters for the subject population using Bayesian information criterion (BIC).

72.-73. (canceled)

74. A kit for identifying a subject having multiple sclerosis (MS) or at risk of developing SPMS, for treatment with an MS therapy, comprising tests for determining the expression levels of two or more genes associated with B cells, two or more genes associated with T cells, two or more genes associated with e-ERYs, and two or more genes associated with GMPs, in a sample.
